(19) United States
(12) Patent Application Publication
(10) Pub. No.: US 2023/0201214 A1
(43) Pub. Date: Jun. 29, 2023

(54) LPXC INHIBITOR, FORMULATIONS, AND USES THEREOF

(71) Applicant: Blacksmith Medicines, Inc., San Diego, CA (US)

(72) Inventors: Min TENG, San Diego, CA (US); Baskar NAMMALWAR, San Diego, CA (US); David T. PUERTA, San Diego, CA (US)

(21) Appl. No.: 18/173,207

(22) Filed: Feb. 23, 2023

Related U.S. Application Data
(63) Continuation of application No. 17/211,025, filed on Mar. 24, 2021, now abandoned.
(60) Provisional application No. 63/153,149, filed on Feb. 24, 2021.
(60) Provisional application No. 62/994,654, filed on Mar. 25, 2020.

(51) Int. Cl. A61K 31/5377; A61K 47/40; A61K 9/00; A61K 47/38

(52) U.S. Cl. CPC A61K 31/5377; A61K 47/40; A61K 9/0019; A61K 9/0053; A61K 47/38

(57) ABSTRACT

Provided herein is an LpxC inhibitor compound, as well as pharmaceutical compositions comprising said compound, and methods of use thereof in the treatment of disease that would benefit from treatment with an LpxC inhibitor, including gram-negative bacterial infections such as urinary tract infections and the like.

### CROSS-REFERENCE

This application is a continuation of U.S. application Ser. No. 17/211,025, filed Mar. 24, 2021, which claims the benefit of U.S. Provisional Patent Application No. 62/994,654, filed Mar. 25, 2020, and U.S. Provisional Patent Application No. 63/153,149, filed Feb. 24, 2021, each of which is incorporated herein by reference in its entirety.

### STATEMENT AS TO FEDERALLY SPONSORED RESEARCH

This invention was made with government support under AI120246 awarded by the National Institutes of Health. The government has certain rights in the invention.

### BACKGROUND

A need exists in the medicinal arts for the effective treatment of illness caused by bacterial infection.

### BRIEF SUMMARY OF THE INVENTION

Provided herein is an LpxC inhibitor compound, as well as pharmaceutical compositions comprising said compound, and methods of use thereof in the treatment of disease that would benefit from treatment with an LpxC inhibitor, including gram-negative bacterial infections such as urinary tract infections and the like.

In one aspect, provided herein is a pharmaceutical composition, comprising:
(i) (S)-1-(3-(5,6-dihydroxypyrimidin-4-yl)-2-(4-((4-(morpholinomethyl)phenyl)ethynyl)phenyl)propyl)azetidine-3-carbonitrile (Compound A):

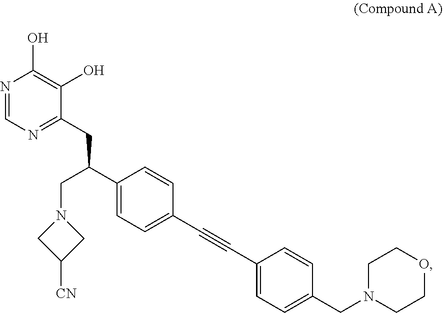 [SMILES: N#CC1CN(C[C@@H](Cc2ncnc(O)c2O)c2ccc(C#Cc3ccc(CN4CCOCC4)cc3)cc2)C1]

or an isotopic variant, tautomer, prodrug, pharmaceutically acceptable salt, solvate, or hydrate thereof; and
(ii) at least one pharmaceutically acceptable excipient.

In some embodiments, provided herein is a pharmaceutical composition, comprising (i) Compound A, or an isotopic variant, tautomer, prodrug, pharmaceutically acceptable salt, solvate, or hydrate thereof; and (ii) at least one pharmaceutically acceptable excipient; wherein the pharmaceutical composition is in a dosage form for dosing or administration by injection.

In some embodiments, the pharmaceutical composition is in a dosage form for intravenous (I.V.) injection or infusion, or intramuscular, subcutaneous, or intradermal injection.

In some embodiments, the pharmaceutical composition is in a dosage form for I.V. injection or infusion.

In some embodiments, the pharmaceutical composition is a solution.

In some embodiments, Compound A, or an isotopic variant, tautomer, prodrug, pharmaceutically acceptable salt, solvate, or hydrate thereof, is crystalline, microcrystalline, amorphous, or lyophilized. In some embodiments, Compound A, or an isotopic variant, tautomer, prodrug, pharmaceutically acceptable salt, solvate, or hydrate thereof, is crystalline. In some embodiments, Compound A, or an isotopic variant, tautomer, prodrug, pharmaceutically acceptable salt, solvate, or hydrate thereof, is amorphous.

In some embodiments, the at least one pharmaceutically acceptable excipient is a co-solvent, oil, surfactant, complexing agent, a solubilizing polymer, a P-gp modulator, a buffering agent, or a combination thereof.

In some embodiments, the at least one pharmaceutically acceptable excipient is a complexing agent. In some embodiments, the complexing agent comprises α-cyclodextrin, p-cyclodextrin, γ-cyclodextrin, methyl-β-cyclodextrin (MPCD), (2-hydroxypropyl)-β-cyclodextrin (HPβCD), sulfobutylether-β-cyclodextrin (SBEβCD), or a combination thereof.

In some embodiments, the pharmaceutical composition comprises sulfobutylether-β-cyclodextrin (SBEβCD). In some embodiments, the pharmaceutical composition comprises from about 1% to about 20% sulfobutylether-β-cyclodextrin (SBEβCD). In some embodiments, the pharmaceutical composition comprises from about 2.5% to about 10% sulfobutylether-p-cyclodextrin (SBEβCD). In some embodiments, the pharmaceutical composition comprises about 2.5%, about 5%, or about 10% sulfobutylether-β-cyclodextrin (SBEβCD).

In some embodiments, the pharmaceutical composition has a pH of from about 2.5 to about 11.0. In some embodiments, the pharmaceutical composition has a pH of from about 2.5 to about 7.0. In some embodiments, the pharmaceutical composition has a pH of from about 4.0 to about 5.0. In some embodiments, the pharmaceutical composition has a pH of about 4.0, about 4.1, about 4.2, about 4.3, about 4.4, about 4.5, about 4.6, about 4.7, about 4.8, about 4.9, or about 5.0.

In some embodiments, the pH of the pharmaceutical composition is adjusted with hydrochloric acid, sodium hydroxide, or a combination thereof.

In some embodiments, the pharmaceutical composition comprises from about 0.1 mg/mL to about 100 mg/mL of Compound A, or an isotopic variant, tautomer, prodrug, pharmaceutically acceptable salt, solvate, or hydrate thereof. In some embodiments, the pharmaceutical composition comprises from about 10 mg/mL to about 50 mg/mL of Compound A, or an isotopic variant, tautomer, prodrug, pharmaceutically acceptable salt, solvate, or hydrate thereof. In some embodiments, the pharmaceutical composition comprises from about 15 mg/mL to about 35 mg/mL of Compound A, or an isotopic variant, tautomer, prodrug, pharmaceutically acceptable salt, solvate, or hydrate thereof. In some embodiments, the pharmaceutical composition comprises about 10 mg/mL, about 15 mg/mL, about 20 mg/mL, about 25 mg/mL, about 30 mg/mL, about 35 mg/mL, about 40 mg/mL, about 45 mg/mL, or about 50 mg/mL of Compound A, or an isotopic variant, tautomer, prodrug, pharmaceutically acceptable salt, solvate, or hydrate thereof.

In some embodiments, the pharmaceutical composition comprises from about 15 mg/g to about 25 mg/g of Compound A, or an isotopic variant, tautomer, prodrug, pharmaceutically acceptable salt, solvate, or hydrate thereof. In some embodiments, the pharmaceutical composition comprises about 10 mg/g, about 15 mg/g, about 20 mg/g, about 25 mg/g, or about 30 mg/g of Compound A, or an isotopic variant, tautomer, prodrug, pharmaceutically acceptable salt, solvate, or hydrate thereof.

In some embodiments, the pharmaceutical composition is stable for up to about 7 days at a temperature of from about 20° C. to 25° C.

In some embodiments, provided herein is a pharmaceutical composition, comprising (i) Compound A, or an isotopic variant, tautomer, prodrug, pharmaceutically acceptable salt, solvate, or hydrate thereof; and (ii) at least one pharmaceutically acceptable excipient; wherein the pharmaceutical composition is in a dosage form for oral dosing or administration.

In some embodiments, the dosage form is a liquid. In some embodiments, the dosage form is a suspension, solution, syrup, or elixir. In some embodiments, the dosage form is a suspension. In some embodiments, the dosage form is a nanosuspension. In some embodiments, the dosage form is a solution. In some embodiments, the dosage form is a tablet or a capsule.

In some embodiments, Compound A, or an isotopic variant, tautomer, prodrug, pharmaceutically acceptable salt, solvate, or hydrate thereof, is crystalline, microcrystalline, amorphous, or lyophilized. In some embodiments, Compound A, or an isotopic variant, tautomer, prodrug, pharmaceutically acceptable salt, solvate, or hydrate thereof, is crystalline. In some embodiments, Compound A, or an isotopic variant, tautomer, prodrug, pharmaceutically acceptable salt, solvate, or hydrate thereof, is amorphous. In some embodiments, Compound A, or an isotopic variant, tautomer, prodrug, pharmaceutically acceptable salt, solvate, or hydrate thereof, is in an amorphous solid dispersion. In some embodiments, the amorphous solid dispersion is a spray dried dispersion. In some embodiments, the amorphous solid dispersion further comprising a cellulose polymer excipient. In some embodiments, the cellulose polymer excipient comprises cellulose acetate phthalate, carboxymethylcellulose sodium, hydroxypropylcellulose acetate succinate, hydroxypropyl methylcellulose 606 (HPMC 606), or hydroxypropyl methylcellulose phthalate (HP-55).

In some embodiments, the at least one pharmaceutically acceptable excipient is a co-solvent, oil, surfactant, complexing agent, a solubilizing polymer, a P-gp modulator, a buffering agent, or a combination thereof.

In some embodiments, the co-solvent comprises PEG200, PEG300, PEG400, PEG600, propylene glycol, ethanol, transcutol, glycerin, or a combination thereof. In some embodiments, the oil comprises sesame oil, soybean oil, vegetable oil, poppyseed oil, safflower oil, peppermint oil, castor oil, oleic acid, maisine CC, capmul MCM, or a combination thereof. In some embodiments, the surfactant comprises polysorbate 20, polysorbate 40, polysorbate 60, polysorbate 80, Gelucire 44/14, vitamin E TPGS, Cremophor RH40, Cremophore RH60, Labrafil M 1944, Labrafil M 2125, Solutol HS 15, or a combination thereof. In some embodiments, the complexing agent comprises α-cyclodextrin, β-cyclodextrin, γ-cyclodextrin, methyl-β-cyclodextrin (MPCD), (2-hydroxypropyl)-β-cyclodextrin (HPβCD), sulfobutylether-$-cyclodextrin (SBEβCD), or a combination thereof. In some embodiments, the solubilizing polymer comprises cellulose acetate phthalate, carboxymethylcellulose sodium, hydroxypropylcellulose acetate succinate, hydroxypropyl methylcellulose 606(HPMC 606), hydroxypropyl methylcellulose phthalate (HP-55), polyvinyl caprolactam-polyvinyl acetate-polyethylene glycol graft copolymer (Soluplus, PCL-PVAc-PEG), or poly(propylene oxide)-poly(ethylene oxide) copolymer (Paloxomer). In some embodiments, the P-gp modulator comprises vitamin E TPGS or cyclosporin A. In some embodiments, the buffering agent comprises phosphate, citrate, lactic acid, proline, histidine, or hydroxide, or a combination thereof.

In some embodiments, the pharmaceutical composition comprises sulfobutylether-p-cyclodextrin (SBEβCD). In some embodiments, the pharmaceutical composition comprises from about 25% to about 50% sulfobutylether-β-cyclodextrin (SBEβCD). In some embodiments, the pharmaceutical composition comprises about 25%, about 30%, about 35%, about 40%, about 45%, or about 50% sulfobutylether-β-cyclodextrin (SBEβCD).

In some embodiments, the pharmaceutical composition comprises HPMC 606. In some embodiments, the pharmaceutical composition comprises from about 0.05% to about 0.5% HPMC606. In some embodiments, the pharmaceutical composition comprises about 0.05%, about 0.075/o, about 0.1%, about 0.15%, about 0.2%, about 0.25%, about 0.3%, about 0.40, or about 0.5% HPMC606.

In some embodiments, the pharmaceutical composition comprises vitamin E TPGS. In some embodiments, the pharmaceutical composition comprises from about 1% to about 10% vitamin E TPGS. In some embodiments, the pharmaceutical composition comprises about 1.0%, about 1.5%, about 2.0%, about 2.5%, about 3%, about 4%, or about 5% vitamin E TPGS.

In some embodiments, the pharmaceutical composition has a pH of from about 2.5 to about 11.0. In some embodiments, the pharmaceutical composition has a pH of from about 2.5 to about 7.0. In some embodiments, the pharmaceutical composition has a pH of from about 3.0 to about 4.5. In some embodiments, the pharmaceutical composition has a pH of about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, or about 4.5.

In some embodiments, the pH of the pharmaceutical composition is adjusted with hydrochloric acid and/or sodium hydroxide.

In some embodiments, the pharmaceutical composition comprises from about 0.1 mg/mL to about 100 mg/mL of Compound A, or an isotopic variant, tautomer, prodrug, pharmaceutically acceptable salt, solvate, or hydrate thereof. In some embodiments, the pharmaceutical composition comprises from about 0.5 mg/mL to about 20 mg/mL of Compound A, or an isotopic variant, tautomer, prodrug, pharmaceutically acceptable salt, solvate, or hydrate thereof. In some embodiments, the pharmaceutical composition comprises from about 1 mg/mL to about 10 mg/mL of Compound A, or an isotopic variant, tautomer, prodrug, pharmaceutically acceptable salt, solvate, or hydrate thereof. In some embodiments, the pharmaceutical composition comprises about 1 mg/mL, about 1.5 mg/mL, about 2 mg/mL, about 2.5 mg/mL, about 3 mg/mL, about 3.5 mg/mL, about 4 mg/mL, about 4.5 mg/mL, or about 5 mg/mL of Compound A, or an isotopic variant, tautomer, prodrug, pharmaceutically acceptable salt, solvate, or hydrate thereof.

In some embodiments, Compound A, or an isotopic variant, tautomer, prodrug, pharmaceutically acceptable salt, solvate, or hydrate thereof, is substantially free of impurities. In some embodiments, Compound A, or an isotopic variant, tautomer, prodrug, pharmaceutically acceptable salt, solvate, or hydrate thereof, is at least about 90% pure, at least about 95% pure, at least about 96% pure, at least about 97% pure, at least about 98% pure, or at least about 99% pure.

In another aspect, disclosed herein is a method of treating a gram-negative bacterial infection in a patient in need thereof comprising administering to the patient the pharmaceutical composition disclosed herein. In some embodiments, the gram-negative bacterial infection is selected from pneumonia, sepsis, cystic fibrosis, intra-abdominal infection, skin infection and urinary tract infection. In some embodiments, the gram-negative bacterial infection is selected from chronic urinary tract infection, complicated urinary tract infection, cystitis, pyelonephritis, urethritis, recurrent urinary tract infections, bladder infections, urethral infections and kidney infections. In some embodiments, the gram-negative bacterial infection is chronic urinary tract infections. In some embodiments, the gram-negative bacterial infection is complicated urinary tract infections. In some embodiments, the composition has no effect on gram-positive bacteria.

In some embodiments, the composition is administered to the patient by I.V. injection or infusion. In other embodiments, the composition is administered to the patient orally.

In some embodiments, the administration is to treat an existing infection. In other embodiments, the administration is provided as prophylaxis.

Articles of manufacture, which include packaging material, the LpxC inhibitory compounds described herein, or an isotopic variant, tautomer, prodrug, pharmaceutically acceptable salt, solvate, or hydrate thereof, within the packaging material, and a label that indicates that the compound or composition, or pharmaceutically acceptable salt, pharmaceutically active metabolite, pharmaceutically acceptable prodrug, or pharmaceutically acceptable solvate thereof, is used for modulating the activity of LpxC, or for the treatment, prevention or amelioration of one or more symptoms of a disease or condition that would benefit from modulation of LpxC activity, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### INCORPORATION BY REFERENCE

All publications, patents, and patent applications mentioned in this specification are herein incorporated by reference for the specific purposes identified herein.

### DETAILED DESCRIPTION OF THE INVENTION

Compound A refers to (S)-1-(3-(5,6-dihydroxypyrimidin-4-yl)-2-(4-((4-(morpholinomethyl)phenyl)ethynyl)phenyl)propyl)azetidine-3-carbonitrile which has the chemical structure shown below.

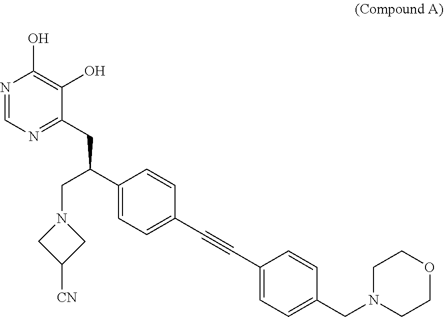 [SMILES: N#CC1CN(C[C@@H](Cc2ncnc(O)c2O)c2ccc(C#Cc3ccc(CN4CCOCC4)cc3)cc2)C1]

Compound A is a potent inhibitor of UDP-{3-O—[(R)-3-hydroxymyristoyl]}-N-acetylglucosamine deacetylase (LpxC). LpxC is an essential enzyme involved in the first committed step in lipid A biosynthesis for gram-negative bacteria. Lipid A is an essential component of the outer membrane of gram-negative bacteria. LpxC is highly conserved across strains of gram-negative bacteria, making LpxC an attractive target to treat gram-negative infections.

Compound A is an LpxC inhibitor that is useful in the methods of treatment described herein. In gram-negative bacterial cell lines, Compound A is a potent inhibitor, exhibiting MIC values of <1 μg/mL against E. coli and K. pneumoniae cell lines. Additionally, Compound A does not inhibit gram-positive bacterial cell lines, such as S. aureus.

The preparation and use of Compound A has been previously described (see, PCT/US2019/052021, which is incorporated by reference in its entirety).

### Definitions

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

The term “acceptable” with respect to a formulation, composition or ingredient, as used herein, means having no persistent detrimental effect on the general health of the subject being treated.

The term “prodrug” is meant to indicate a compound that is, in some embodiments, converted under physiological conditions or by solvolysis to a biologically active compound described herein. Thus, the term “prodrug” refers to a precursor of a biologically active compound that is pharmaceutically acceptable. A prodrug is typically inactive when administered to a subject, but is converted in vivo to an active compound, for example, by hydrolysis. The prodrug compound often offers advantages of solubility, tissue compatibility or delayed release in a mammalian organism (see, e.g., Bundgard, H., Design of Prodrugs (1985), pp. 7 9, 21 24 (Elsevier, Amsterdam). A discussion of prodrugs is provided in Higuchi, T., et al., “Pro drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987. The term “prodrug” is also meant to include any covalently bonded carriers, which release the active compound in vivo when such prodrug is administered to a mammalian subject. Prodrugs of an active compound, as described herein, are prepared by modifying functional groups present in the active compound in such a way that the modifications are cleaved, either in routine manipulation or in vivo, to the parent active compound. Prodrugs include compounds wherein a hydroxy, amino or mercapto group is bonded to any group that, when the prodrug of the active compound is administered to a mammalian subject, cleaves to form a free hydroxy, free amino or free mercapto group, respectively. Examples of prodrugs include, but are not limited to, acetate, formate and benzoate derivatives of alcohol or amine functional groups in the active compounds and the like.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an antagonist.

The terms “administer,” “administering”, “administration,” and the like, as used herein, refer to the methods that may be used to enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally. In some embodiments, the compounds and compositions described herein are administered intravenously.

The terms “co-administration” or the like, as used herein, are meant to encompass administration of the selected therapeutic agents to a single patient, and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. the LpxC inhibitory compound disclosed herein, or an isotopic variant, tautomer, prodrug, pharmaceutically acceptable salt, solvate, or hydrate thereof, and a co-agent, are both administered to a patient simultaneously in the form of a single entity or dosage. The term “non-fixed combination” means that the active ingredients, e.g. the LpxC inhibitory compound disclosed herein, or an isotopic variant, tautomer, prodrug, pharmaceutically acceptable salt, solvate, or hydrate thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “article of manufacture” and “kit” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Further Forms of Compound A

“Pharmaceutically acceptable,” as used herein, refers a material, such as a carrier or diluent, which does not abrogate the biological activity or properties of the compound, and is relatively nontoxic, i.e., the material is administered to an individual without causing undesirable biological effects or interacting in a deleterious manner with any of the components of the composition in which it is contained.

The term “pharmaceutically acceptable salt” refers to a form of a therapeutically active agent that consists of a cationic form of the therapeutically active agent in combination with a suitable anion, or in alternative embodiments, an anionic form of the therapeutically active agent in combination with a suitable cation. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich:Wiley-VCH/VHCA, 2002. Pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability can be manipulated as one aspect of delayed and sustained release behaviours. Also, because the salt-forming molecule can be in equilibrium with a neutral form, passage through biological membranes can be adjusted.

In some embodiments, pharmaceutically acceptable salts are obtained by reacting a compound disclosed herein with an acid. In some embodiments, the compound disclosed herein (i.e. free base form) is basic and is reacted with an organic acid or an inorganic acid. Inorganic acids include, but are not limited to, hydrochloric acid, hydrobromic acid, sulfuric acid, phosphoric acid, nitric acid, and metaphosphoric acid. Organic acids include, but are not limited to, 1-hydroxy-2-naphthoic acid; 2,2-dichloroacetic acid; 2-hydroxyethanesulfonic acid; 2-oxoglutaric acid; 4-acetamidobenzoic acid; 4-aminosalicylic acid; acetic acid; adipic acid; ascorbic acid (L); aspartic acid (L); benzenesulfonic acid; benzoic acid; camphoric acid (+); camphor-10-sulfonic acid (+); capric acid (decanoic acid); caproic acid (hexanoic acid); caprylic acid (octanoic acid); carbonic acid; cinnamic acid; citric acid; cyclamic acid; dodecylsulfuric acid; ethane-1,2-disulfonic acid; ethanesulfonic acid; formic acid; fumaric acid; galactaric acid; gentisic acid; glucoheptonic acid (D); gluconic acid (D); glucuronic acid (D); glutamic acid; glutaric acid; glycerophosphoric acid; glycolic acid; hippuric acid; isobutyric acid; lactic acid (DL); lactobionic acid; lauric acid; maleic acid; malic acid (−L); malonic acid; mandelic acid (DL); methanesulfonic acid; naphthalene-1,5-disulfonic acid; naphthalene-2-sulfonic acid; nicotinic acid; oleic acid; oxalic acid; palmitic acid; pamoic acid; phosphoric acid; proprionic acid; pyroglutamic acid (−L); salicylic acid; sebacic acid; stearic acid; succinic acid; sulfuric acid; tartaric acid (+L); thiocyanic acid; toluenesulfonic acid (p); and undecylenic acid.

In some embodiments, pharmaceutically acceptable salts are obtained by reacting a compound disclosed herein with a base. In some embodiments, the compound disclosed herein is acidic and is reacted with a base. In such situations, an acidic proton of the compound disclosed herein is replaced by a metal ion, e.g., lithium, sodium, potassium, magnesium, calcium, or an aluminum ion. In some cases, compounds described herein coordinate with an organic base, such as, but not limited to, ethanolamine, diethanolamine, triethanolamine, tromethamine, meglumine, N-methylglucamine, dicyclohexylamine, tris(hydroxymethyl)methylamine. In other cases, compounds described herein form salts with amino acids such as, but not limited to, arginine, lysine, and the like. Acceptable inorganic bases used to form salts with compounds that include an acidic proton, include, but are not limited to, aluminum hydroxide, calcium hydroxide, potassium hydroxide, sodium carbonate, potassium carbonate, sodium hydroxide, lithium hydroxide, and the like. In some embodiments, the compounds provided herein are prepared as a sodium salt, calcium salt, potassium salt, magnesium salt, meglumine salt, N-methylglucamine salt or ammonium salt.

It should be understood that a reference to a pharmaceutically acceptable salt includes the solvent addition forms. In some embodiments, solvates contain either stoichiometric or non-stoichiometric amounts of a solvent, and are formed during the process of crystallization with pharmaceutically acceptable solvents such as water, ethanol, and the like. Hydrates are formed when the solvent is water, or alcoholates are formed when the solvent is alcohol. Solvates of compounds described herein are conveniently prepared or formed during the processes described herein. In addition, the compounds provided herein optionally exist in unsolvated as well as solvated forms.

Therapeutic agents that are administrable to mammals, such as humans, must be prepared by following regulatory guidelines. Such government regulated guidelines are referred to as Good Manufacturing Practice (GMP). GMP guidelines outline acceptable contamination levels of active therapeutic agents, such as, for example, the amount of residual solvent in the final product. Preferred solvents are those that are suitable for use in GMP facilities and consistent with industrial safety concerns. Categories of solvents are defined in, for example, the International Conference on Harmonization of Technical Requirements for Registration of Pharmaceuticals for Human Use (ICH), “Impurities: Guidelines for Residual Solvents, Q3C(R3) (November 2005).

Solvents are categorized into three classes. Class 1 solvents are toxic and are to be avoided. Class 2 solvents are solvents to be limited in use during the manufacture of the therapeutic agent. Class 3 solvents are solvents with low toxic potential and of lower risk to human health. Data for Class 3 solvents indicate that they are less toxic in acute or short-term studies and negative in genotoxicity studies.

Class 1 solvents, which are to be avoided, include: benzene; carbon tetrachloride; 1,2-dichloroethane; 1,1-dichloroethene; and 1,1,1-trichloroethane.

Examples of Class 2 solvents are: acetonitrile, chlorobenzene, chloroform, cyclohexane, 1,2-dichloroethene, dichloromethane, 1,2-dimethoxyethane, N,N-dimethylacetamide, N,N-dimethylformamide, 1,4-dioxane, 2-ethoxyethanol, ethyleneglycol, formamide, hexane, methanol, 2-methoxyethanol, methylbutyl ketone, methylcyclohexane, N-methylpyrrolidine, nitromethane, pyridine, sulfolane, tetralin, toluene, 1,1,2-trichloroethene and xylene.

Class 3 solvents, which possess low toxicity, include: acetic acid, acetone, anisole, 1-butanol, 2-butanol, butyl acetate, tert-butylmethyl ether (MTBE), cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran.

Residual solvents in active pharmaceutical ingredients (APIs) originate from the manufacture of API. In some cases, the solvents are not completely removed by practical manufacturing techniques. Appropriate selection of the solvent for the synthesis of APIs may enhance the yield, or determine characteristics such as crystal form, purity, and solubility. Therefore, the solvent is a critical parameter in the synthetic process.

In some embodiments, compositions comprising Compound A, comprise an organic solvent(s). In some embodiments, compositions comprising Compound A include a residual amount of an organic solvent(s). In some embodiments, compositions comprising Compound A comprise a residual amount of a Class 3 solvent. In some embodiments, the Class 3 solvent is selected from the group consisting of acetic acid, acetone, anisole, I-butanol, 2-butanol, butyl acetate, tert-butylmethyl ether, cumene, dimethyl sulfoxide, ethanol, ethyl acetate, ethyl ether, ethyl formate, formic acid, heptane, isobutyl acetate, isopropyl acetate, methyl acetate, 3-methyl-1-butanol, methylethyl ketone, methylisobutyl ketone, 2-methyl-1-propanol, pentane, 1-pentanol, 1-propanol, 2-propanol, propyl acetate, and tetrahydrofuran. In some embodiments, the Class 3 solvent is selected from ethyl acetate, isopropyl acetate, tert-butylmethylether, heptane, isopropanol, and ethanol.

In some embodiments, the compositions comprising Compound A include a detectable amount of an organic solvent. In some embodiments, the organic solvent is a Class 3 solvent.

In other embodiments are compositions comprising Compound A wherein the composition comprises a detectable amount of solvent that is less than about 1%, wherein the solvent is selected from acetone, 1,2-dimethoxyethane, acetonitrile, ethyl acetate, tetrahydrofuran, methanol, ethanol, heptane, and 2-propanol. In a further embodiment are compositions comprising Compound A wherein the composition comprises a detectable amount of solvent which is less than about 5000 ppm. In yet a further embodiment are compositions comprising Compound A, wherein the detectable amount of solvent is less than about 5000 ppm, less than about 4000 ppm, less than about 3000 ppm, less than about 2000 ppm, less than about 1000 ppm, less than about 500 ppm, or less than about 100 ppm.

The methods and formulations described herein include the use of N-oxides (if appropriate), or pharmaceutically acceptable salts of compounds having the structure disclosed herein, as well as active metabolites of these compounds having the same type of activity.

In some embodiments, sites on the organic radicals (e.g. alkyl groups, aromatic rings) of compounds disclosed herein are susceptible to various metabolic reactions. Incorporation of appropriate substituents on the organic radicals will reduce, minimize or eliminate this metabolic pathway. In specific embodiments, the appropriate substituent to decrease or eliminate the susceptibility of the aromatic ring to metabolic reactions is, by way of example only, a halogen, deuterium, an alkyl group, a haloalkyl group, or a deuteroalkyl group.

In another embodiment, the compounds described herein are labeled isotopically (e.g. with a radioisotope) or by another other means, including, but not limited to, the use of chromophores or fluorescent moieties, bioluminescent labels, or chemiluminescent labels.

Compounds described herein include isotopically-labeled compounds, which are identical to those recited in the various formulae and structures presented herein, but for the fact that one or more atoms are replaced by an atom having an atomic mass or mass number different from the atomic mass or mass number usually found in nature. Examples of isotopes that can be incorporated into the present compounds include isotopes of hydrogen, carbon, nitrogen, oxygen, sulfur, fluorine chlorine, iodine, phosphorus, such as, for example, ²H, ³H, ¹³C, ¹⁴C, ¹⁵N, ¹⁸O, ¹⁷O, ³⁵S, ¹⁸F, ³⁶Cl, ¹²³I, ¹²⁴I, ¹²⁵I, ¹³¹I, ³²P and ³³P. In one aspect, isotopically-labeled compounds described herein, for example those into which radioactive isotopes such as ³H and ¹⁴C are incorporated, are useful in drug and/or substrate tissue distribution assays. In one aspect, substitution with isotopes such as deuterium affords certain therapeutic advantages resulting from greater metabolic stability, such as, for example, increased in vivo half-life or altered metabolic pathways to reduce undesirable metabolites or reduced dosage requirements.

In some embodiments, one or more hydrogen atoms on Compound A are replaced with deuterium. In some embodiments, substitution with deuterium affords certain therapeutic advantages resulting from greater metabolic stability, such as, for example, increased in vivo half-life or reduced dosage requirements.

In one aspect, described is a compound with the following structure:

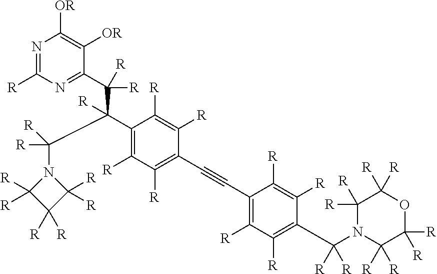

wherein,
each R is independently selected from hydrogen or deuterium,
or an isotopic variant, tautomer, pharmaceutically acceptable salt, solvate, or hydrate thereof.

In some embodiments, the compounds disclosed herein possess one or more stereocenters and each stereocenter exists independently in either the R or S configuration. For example, in some embodiments, the compound disclosed herein exists in the R configuration when one stereocenter is present. In other embodiments, the compound disclosed herein exists in the S configuration when one stereocenter is present. In some embodiments, the compound disclosed herein exists in the RR configuration when two stereocenters are present. In some embodiments, the compound disclosed herein exists in the RS configuration when two stereocenters are present. In some embodiments, the compound disclosed herein exists in the SS configuration when two stereocenters are present. In some embodiments, the compound disclosed herein exists in the SR configuration when two stereocenters are present.

The compounds presented herein include all diastereomeric, individual enantiomers, atropisomers, and epimeric forms as well as the appropriate mixtures thereof. The compounds and methods provided herein include all cis, trans, syn, anti, entgegen (E), and zusammen (Z) isomers as well as the appropriate mixtures thereof.

Individual stereoisomers are obtained, if desired, by methods such as, stereoselective synthesis and/or the separation of stereoisomers by chiral chromatographic columns or the separation of diastereomers by either non-chiral or chiral chromatographic columns or crystallization and recrystallization in a proper solvent or a mixture of solvents. In certain embodiments, compounds disclosed herein are prepared as their individual stereoisomers by reacting a racemic mixture of the compound with an optically active resolving agent to form a pair of diastereoisomeric compounds/salts, separating the diastereomers and recovering the optically pure individual enantiomers. In some embodiments, resolution of individual enantiomers of compounds disclosed herein is carried out using covalent diastereomeric derivatives of the compounds described herein. In another embodiment, diastereomers of compounds disclosed herein are separated by separation/resolution techniques based upon differences in solubility. In other embodiments, separation of stereoisomers of compounds disclosed herein is performed by chromatography or by the forming diastereomeric salts and separation by recrystallization, or chromatography, or any combination thereof. Jean Jacques, Andre Collet, Samuel H. Wilen, “Enantiomers, Racemates and Resolutions”, John Wiley And Sons, Inc., 1981. In some embodiments, stereoisomers are obtained by stereoselective synthesis. Separation of individual enantiomers from a racemic mixture is possible by the use of chiral supercritical fluid chromatography (SFC) or chiral high performance liquid chromatography (HPLC). In some embodiments, enantiomers described herein are separated from each other by the use of chiral SFC or chiral HPLC. In some embodiments, compounds disclosed herein that include one or more chiral centers (e.g. compounds disclosed herein that include the moiety trans-octahydro-1H-pyrido[3,4-b]morpholin-6-yl) are separated into individual enantiomers using chiral SFC or chiral HPLC. A wide variety of conditions and suitable columns are available.

Daicel polysaccharide chiral stationary phases (CSPs) are among the columns used for chiral SFC separations. In some embodiments, Daicel analytical immobilised and coated CHIRALPAK and CHIRALCEL HPLC columns can be used for SFC analysis.

In some embodiments, screening for the suitability of using a SFC column is performed on the four main immobilised phases (CHIRALPAK IA, IB, IC and ID) and the four main coated columns (CHIRALPAK AD and AS and CHIRALCEL OD and OJ), with varying concentrations of organic modifier. A variety of column phases are available, including but not limited to OD and OJ, OX and OZ chlorinated phases, and a range of complementary cellulose based CHIRALCEL phases including OA, OB, OC, OF, OG and OK.

Non-limiting examples of chiral selectors contemplated for use in the separation of enantiomers include amylose tris (3, 5-dimethylphenylcarbamate), cellulose tris (3, 5-dimethylphenylcarbamate), cellulose tris (3, 5-dichlorophenylcarbamate), amylose tris (3-chlorophenylcarbamate), amylosetris (3, 5-dichlorophenylcarbamate), amylosetris (3-chloro, 4-methylphenylcarbamate), amylose tris ((S)-alpha-methylbenzylcarbamate), amylose tris (5-chloro-2-methylphenylcarbamate), cellulose tris (4-methylbenzoate), cellulose tris (4-chloro-3-methylphenylcarbamate), and cellulose tris (3-chloro-4-methylphenylcarbamate).

Non-limiting examples of chiral columns contemplated for use in the separation of enantiomers include CHIRALPAK IA SFC, CHIRALPAK AD-H SFC, CHIRALPAK IB SFC, CHIRALCEL OD-H SFC, CHIRALPAK IC SFC, CHIRALPAK ID SFC, CHIRALPAK IE SFC, CHIRALPAK IF SFC, CHIRALPAK AZ-H SFC, CHIRALPAK AS-H SFC, CHIRALPAK AY-H SFC, CHIRALCEL OJ-H SFC, CHIRALCEL OX-H SFC, and CHIRALCEL OZ-H SFC.

In additional or further embodiments, the compounds described herein are metabolized upon administration to an organism in need to produce a metabolite that is then used to produce a desired effect, including a desired therapeutic effect.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

### Pharmaceutical Compositions

In certain embodiments, the heterocyclic LpxC inhibitory compound as described herein is administered as a pure chemical. In other embodiments, the heterocyclic LpxC inhibitory compound described herein is combined with a pharmaceutically suitable or acceptable carrier (also referred to herein as a pharmaceutically suitable (or acceptable) excipient, physiologically suitable (or acceptable) excipient, or physiologically suitable (or acceptable) carrier) selected on the basis of a chosen route of administration and standard pharmaceutical practice as described, for example, in Remington: The Science and Practice of Pharmacy (Gennaro, 21^{st} Ed. Mack Pub. Co., Easton, Pa. (2005)).

Provided herein is a pharmaceutical composition comprising the LpxC inhibitory compound described herein, or an isotopic variant, tautomer, prodrug, pharmaceutically acceptable salt, solvate, or hydrate thereof, together with one or more pharmaceutically acceptable carriers. The carrier(s) (or excipient(s)) is acceptable or suitable if the carrier is compatible with the other ingredients of the composition and not deleterious to the recipient (i.e., the subject or patient) of the composition.

In one aspect, provided herein is a pharmaceutical composition, comprising:
(i) (S)-1-(3-(5,6-dihydroxypyrimidin-4-yl)-2-(4-((4-(morpholinomethyl)phenyl)ethynyl)phenyl)propyl)azetidine-3-carbonitrile (Compound A):

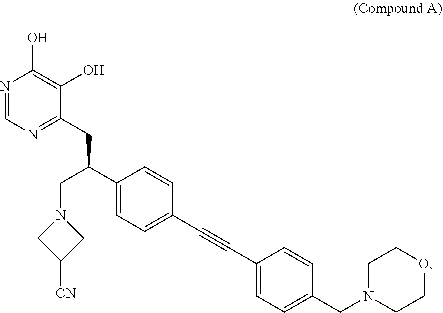 [SMILES: N#CC1CN(C[C@@H](Cc2ncnc(O)c2O)c2ccc(C#Cc3ccc(CN4CCOCC4)cc3)cc2)C1]

or an isotopic variant, tautomer, prodrug, pharmaceutically acceptable salt, solvate, or hydrate thereof; and
(ii) at least one pharmaceutically acceptable excipient.

In some embodiments, the pharmaceutical composition is in a dosage form for dosing or administration by injection. In some embodiments, the pharmaceutical composition is in a dosage form for intravenous (I.V.) injection or infusion, or intramuscular, subcutaneous, or intradermal injection. In some embodiments, the pharmaceutical composition is in a dosage form for I.V. injection or infusion. In some embodiments, the pharmaceutical composition is a solution.

In some embodiments, the pharmaceutical composition is in a dosage form for oral dosing or administration. In some embodiments, the dosage form is a liquid. In some embodiments, the dosage form is a suspension, solution, syrup, or elixir. In some embodiments, the dosage form is a suspension. In some embodiments, the dosage form is a nanosuspension. In some embodiments, the dosage form is a solution. In other embodiments, the dosage form is a tablet or a capsule.

In some embodiments, Compound A, or an isotopic variant, tautomer, prodrug, pharmaceutically acceptable salt, solvate, or hydrate thereof, is crystalline, microcrystalline, amorphous, or lyophilized. In some embodiments, Compound A, or an isotopic variant, tautomer, prodrug, pharmaceutically acceptable salt, solvate, or hydrate thereof, is crystalline. In some embodiments, Compound A, or an isotopic variant, tautomer, prodrug, pharmaceutically acceptable salt, solvate, or hydrate thereof, is amorphous. In some embodiments, Compound A, or an isotopic variant, tautomer, prodrug, pharmaceutically acceptable salt, solvate, or hydrate thereof, is in an amorphous solid dispersion. In some embodiments, the amorphous solid dispersion is a spray dried dispersion. In some embodiments, the amorphous solid dispersion further comprising a cellulose polymer excipient. In some embodiments, the cellulose polymer excipient comprises cellulose acetate phthalate, carboxymethylcellulose sodium, hydroxy propylcellulose acetate succinate, hydroxypropyl methylcellulose 606 (HPMC 606), or hydroxypropyl methylcellulose phthalate (HP-55). In some embodiments, the cellulose polymer excipient comprises HPMC 606 or HP-55.

In some embodiments, the at least one pharmaceutically acceptable excipient is a co-solvent, oil, surfactant, complexing agent, a solubilizing polymer, a P-gp modulator, a buffering agent, or a combination thereof.

In some embodiments, the co-solvent comprises PEG200, PEG300, PEG400, PEG600, propylene glycol, ethanol, transcutol, glycerin, or a combination thereof. In some embodiments, the cosolvent comprises PEG 400, propylene glycol, transcutol, or a combination thereof.

In some embodiments, the oil comprises sesame oil, soybean oil, vegetable oil, poppyseed oil, safflower oil, peppermint oil, castor oil, oleic acid, maisine CC, capmul MCM, or a combination thereof. In some embodiments, the oil comprises soybean oil, oleic acid, maisine CC, peppermint oil, capmul MCM, or a combination thereof.

In some embodiments, the surfactant comprises polysorbate 20, polysorbate 40, polysorbate 60, polysorbate 80, Gelucire 44/14, vitamin E TPGS, Cremophor RH40, Cremophore RH60, Labrafil M 1944, Labrafil M 2125, Solutol HS 15, or a combination thereof.

In some embodiments, the surfactant comprises Gelucire 44/14, vitamin E TPGS, polysorbate 20, polysorbate 80, Labrafil M 1944, Solutol HS 15, or a combination thereof.

In some embodiments, the complexing agent comprises α-cyclodextrin, β-cyclodextrin, γ-cyclodextrin, methyl-β-cyclodextrin (MPCD), (2-hydroxypropyl)-β-cyclodextrin (HPPCD), sulfobutylether-β-cyclodextrin (SBEβCD), or a combination thereof. In some embodiments, the complexing agent comprises HPβCD, SBEβCD, or a combination thereof. In some embodiments, the complexing agent comprises SBEβCD.

In some embodiments, the solubilizing polymer comprises cellulose acetate phthalate, carboxymethylcellulosesodium, hydroxypropylcelluloseacetate succinate, hydroxypropyl methylcellulose 606(HPMC 606), hydroxypropyl methylcellulose phthalate (HP-55), polyvinyl caprolactam-polyvinyl acetate-polyethylene glycol graft copolymer (Soluplus, PCL-PVAc-PEG), or poly(propylene oxide)-poly(ethylene oxide) copolymer (Paloxomer). In some embodiments, the solubilizing polymer comprises HPMC 606.

In some embodiments, the P-gp modulator comprises vitamin E TPGS or cyclosporin A. In some embodiments, the P-gp modulator comprises vitamin E TPGS.

In some embodiments, the buffering agent comprises phosphate, citrate, lactic acid, proline, histidine, or hydroxide, or a combination thereof. In some embodiments, the buffering agent comprises citrate, lactic acid, or a combination thereof. In some embodiments, the buffering agent comprises citrate.

In some embodiments, the pharmaceutical composition has a pH of from about 2.5 to about 11.0. In some embodiments, the pharmaceutical composition has a pH of from about 2.5 to about 7.0. In some embodiments, the pharmaceutical composition has a pH of from about 4.0 to about 5.0. In some embodiments, the pharmaceutical composition has a pH of from about 3.0 to about 4.5.

In some embodiments, the pH of the pharmaceutical composition is adjusted with hydrochloric acid, sodium hydroxide, or a combination thereof. In some embodiments, the pH of the pharmaceutical composition is adjusted with hydrochloric acid. In some embodiments, the pH of the pharmaceutical composition is adjusted with sodium hydroxide.

In some embodiments, the pharmaceutical composition comprises from about 0.1 mg/mL to about 100 mg/mL of Compound A, or an isotopic variant, tautomer, prodrug, pharmaceutically acceptable salt, solvate, or hydrate thereof. In some embodiments, the pharmaceutical composition comprises from about 10 mg/mL to about 50 mg/mL of Compound A, or an isotopic variant, tautomer, prodrug, pharmaceutically acceptable salt, solvate, or hydrate thereof. In some embodiments, the pharmaceutical composition comprises from about 15 mg/mL to about 35 mg/mL of Compound A, or an isotopic variant, tautomer, prodrug, pharmaceutically acceptable salt, solvate, or hydrate thereof. In some embodiments, the pharmaceutical composition comprises from about 1 mg/mL to about 10 mg/mL of Compound A, or an isotopic variant, tautomer, prodrug, pharmaceutically acceptable salt, solvate, or hydrate thereof.

In some embodiments, Compound A, or an isotopic variant, tautomer, prodrug, pharmaceutically acceptable salt, solvate, or hydrate thereof, is free of impurities. In some embodiments, Compound A, or an isotopic variant, tautomer, prodrug, pharmaceutically acceptable salt, solvate, or hydrate thereof, is essentially free of impurities. In some embodiments, Compound A, or an isotopic variant, tautomer, prodrug, pharmaceutically acceptable salt, solvate, or hydrate thereof, is substantially free of impurities.

In some embodiments, Compound A, or an isotopic variant, tautomer, prodrug, pharmaceutically acceptable salt, solvate, or hydrate thereof, is at least about 90% pure, at least about 95% pure, at least about 96% pure, at least about 97% pure, at least about 98% pure, or at least about 99% pure. In some embodiments, Compound A, or an isotopic variant, tautomer, prodrug, pharmaceutically acceptable salt, solvate, or hydrate thereof, is at least about 99.0% pure, at least about 99.1% pure, at least about 99.2% pure, at least about 99.3% pure, at least about 99.4% pure, at least about 99.5% pure, at least about 99.6% pure, at least about 99.7% pure, at least about 99.8% pure, or at least about 99.9% pure.

In some embodiments, provided herein is a pharmaceutical composition, comprising:
(i) (S)-1-(3-(5,6-dihydroxypyrimidin-4-yl)-2-(4-((4-(morpholinomethyl)phenyl)ethynyl)phenyl)propyl)azetidine-3-carbonitrile (Compound A):

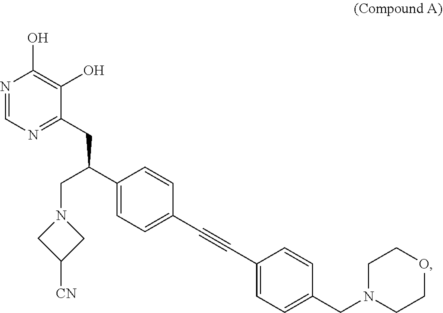 [SMILES: N#CC1CN(C[C@@H](Cc2ncnc(O)c2O)c2ccc(C#Cc3ccc(CN4CCOCC4)cc3)cc2)C1]

or an isotopic variant, tautomer, prodrug, pharmaceutically acceptable salt, solvate, or hydrate thereof; and
(ii) at least one pharmaceutically acceptable excipient;
wherein the pharmaceutical composition is in a dosage form for dosing or administration by injection

In some embodiments, the pharmaceutical composition is in a dosage form for intravenous (I.V.) injection or infusion, or intramuscular, subcutaneous, or intradermal injection. In some embodiments, the pharmaceutical composition is in a dosage form for I.V. injection or infusion.

In some embodiments, the pharmaceutical composition is a solution.

In some embodiments, Compound A, or an isotopic variant, tautomer, prodrug, pharmaceutically acceptable salt, solvate, or hydrate thereof, is crystalline, microcrystalline, amorphous, or lyophilized. In some embodiments, Compound A, or an isotopic variant, tautomer, prodrug, pharmaceutically acceptable salt, solvate, or hydrate thereof, is crystalline. In some embodiments, Compound A, or an isotopic variant, tautomer, prodrug, pharmaceutically acceptable salt, solvate, or hydrate thereof, is amorphous.

In some embodiments, the at least one pharmaceutically acceptable excipient is a co-solvent, oil, surfactant, complexing agent, a solubilizing polymer, a P-gp modulator, a buffering agent, or a combination thereof. In some embodiments, the at least one pharmaceutically acceptable excipient is a co-solvent, complexing agent, a buffering agent, or a combination thereof. In some embodiments, the at least one pharmaceutically acceptable excipient is a complexing agent.

In some embodiments, the complexing agent comprises α-cyclodextrin, β-cyclodextrin, γ-cyclodextrin, methyl-β-cyclodextrin (MPCD), (2-hydroxypropyl)-β-cyclodextrin (HPPCD), sulfobutylether-β-cyclodextrin (SBEβCD), or a combination thereof. In some embodiments, the complexing agent is HPβCD or SBEβCD. In some embodiments, the complexing agent is SBEβCD.

In some embodiments, use of a complexing agent (such as a cyclodextrin) provides high solubility of Compound A. In some embodiments, use of a complexing agent provides higher solubility of Compound A than a formulation without a complexing agent.

In some embodiments, use of a complexing agent (such as a cyclodextrin) provides slower release of Compound A upon administration. In some embodiments, use of a complexing agent provides slower release of Compound A into the blood stream upon administration.

In some embodiments, use of a complexing agent (such as a cyclodextrin) provides higher drug loading, or dosing concentration. In some embodiments, when a complexing agent is absent, there is precipitation of the drug upon administration. In some embodiments, when a complexing agent is absent, there is precipitation of the drug upon dilution into PBS buffer (pH 7.4), which mimics or models the blood. In some embodiments, when a complexing agent is absent, there is higher protein binding of the drug upon administration.

In some embodiments, the pharmaceutical composition comprises sulfobutylether-p-cyclodextrin (SBEβCD). In some embodiments, the pharmaceutical composition comprises from about 0.1% to about 50% SBEβCD. In some embodiments, the pharmaceutical composition comprises from about 1% to about 20% SBEβCD. In some embodiments, the pharmaceutical composition comprises from about 2.5% to about 10% SBEβCD. In some embodiments, the pharmaceutical composition comprises about 2.5%, about 5%, about 10%, about 15%, or about 20% SBEβCD. In some embodiments, the pharmaceutical composition comprises about 2.5%, about 5%, or about 10% SBEβCD. In some embodiments, the pharmaceutical composition comprises about 2.5% SBEβCD. In some embodiments, the pharmaceutical composition comprises about 5% SBEβCD. In some embodiments, the pharmaceutical composition comprises about 10% SBEβCD. In some embodiments, the pharmaceutical composition comprises from 1% to 20% SBEβCD. In some embodiments, the pharmaceutical composition comprises from 2.5% to 10% SBEβCD. In some embodiments, the pharmaceutical composition comprises 2.5%, 5%, 10% SBEβCD, 15%, or 20%. In some embodiments, the pharmaceutical composition comprises 2.5%, 5%, or 10% SBEβCD. In some embodiments, the pharmaceutical composition comprises 2.5% SBEβCD. In some embodiments, the pharmaceutical composition comprises 5% SBEβCD. In some embodiments, the pharmaceutical composition comprises 10% SBEβCD.

In some embodiments, the pharmaceutical composition has a pH of from about 2.5 to about 11.0. In some embodiments, the pharmaceutical composition has a pH of from about 2.5 to about 7.0. In some embodiments, the pharmaceutical composition has a pH of from about 4.0 to about 5.0. In some embodiments, the pharmaceutical composition has a pH of about 4.0, about 4.1, about 4.2, about 4.3, about 4.4, about 4.5, about 4.6, about 4.7, about 4.8, about 4.9, or about 5.0. In some embodiments, the pharmaceutical composition has a pH of from 2.5 to 11.0. In some embodiments, the pharmaceutical composition has a pH of from 2.5 to 7.0. In some embodiments, the pharmaceutical composition has a pH of from 4.0 to 5.0. In some embodiments, the pharmaceutical composition has a pH of 4.0, 4.1, 4.2, 4.3, 4.4, 4.5, 4.6, 4.7, 4.8, 4.9, or 5.0. In some embodiments, the pharmaceutical composition has a pH of 4.10, 4.11, 4.12, 4.13, 4.14, 4.15, 4.16, 4.17, 4.18, 4.19, 4.20, 4.21, 4.22, 4.23, 4.24, 4.25, 4.26, 4.27, 4.28, 4.29, or 4.30.

In some embodiments, the pH of the pharmaceutical composition is adjusted with hydrochloric acid, sodium hydroxide, or a combination thereof. In some embodiments, the pH of the pharmaceutical composition is adjusted with hydrochloric acid. In some embodiments, the pH of the pharmaceutical composition is adjusted with sodium hydroxide.

In some embodiments, the pharmaceutical composition comprises from about 0.1 mg/mL to about 100 mg/mL of Compound A, or an isotopic variant, tautomer, prodrug, pharmaceutically acceptable salt, solvate, or hydrate thereof. In some embodiments, the pharmaceutical composition comprises from about 10 mg/mL to about 50 mg/mL of Compound A, or an isotopic variant, tautomer, prodrug, pharmaceutically acceptable salt, solvate, or hydrate thereof. In some embodiments, the pharmaceutical composition comprises from about 15 mg/mL to about 35 mg/mL of Compound A, or an isotopic variant, tautomer, prodrug, pharmaceutically acceptable salt, solvate, or hydrate thereof. In some embodiments, the pharmaceutical composition comprises about 10 mg/mL, about 15 mg/mL, about 20 mg/mL, about 25 mg/mL, about 30 mg/mL, about 35 mg/mL, about 40 mg/mL, about 45 mg/mL, or about 50 mg/mL of Compound A, or an isotopic variant, tautomer, prodrug, pharmaceutically acceptable salt, solvate, or hydrate thereof. In some embodiments, the pharmaceutical composition comprises from 10 mg/mL to 50 mg/mL of Compound A, or an isotopic variant, tautomer, prodrug, pharmaceutically acceptable salt, solvate, or hydrate thereof. In some embodiments, the pharmaceutical composition comprises from 15 mg/mL to 35 mg/mL of Compound A, or an isotopic variant, tautomer, prodrug, pharmaceutically acceptable salt, solvate, or hydrate thereof. In some embodiments, the pharmaceutical composition comprises 10 mg/mL, 15 mg/mL, 20 mg/mL, 25 mg/mL, 30 mg/mL, 35 mg/mL, 40 mg/mL, 45 mg/mL, or 50 mg/mL of Compound A, or an isotopic variant, tautomer, prodrug, pharmaceutically acceptable salt, solvate, or hydrate thereof.

In some embodiments, the pharmaceutical composition comprises from about 0.1 mg/g to about 100 mg/g of Compound A, or an isotopic variant, tautomer, prodrug, pharmaceutically acceptable salt, solvate, or hydrate thereof. In some embodiments, the pharmaceutical composition comprises from about 10 mg/g to about 50 mg/g of Compound A, or an isotopic variant, tautomer, prodrug, pharmaceutically acceptable salt, solvate, or hydrate thereof. In some embodiments, the pharmaceutical composition comprises from about 15 mg/g to about 25 mg/g of Compound A, or an isotopic variant, tautomer, prodrug, pharmaceutically acceptable salt, solvate, or hydrate thereof. In some embodiments, the pharmaceutical composition comprises about 10 mg/g, about 15 mg/g, about 20 mg/g, about 25 mg/g, or about 30 mg/g, of Compound A, or an isotopic variant, tautomer, prodrug, pharmaceutically acceptable salt, solvate, or hydrate thereof. In some embodiments, the pharmaceutical composition comprises about 15 mg/g, about 16 mg/g, about 17 mg/g, about 18 mg/g, about 19 mg/g, or about 20 mg/g, of Compound A, or an isotopic variant, tautomer, prodrug, pharmaceutically acceptable salt, solvate, or hydrate thereof. In some embodiments, the pharmaceutical composition comprises from 10 mg/g to 50 mg/g of Compound A, or an isotopic variant, tautomer, prodrug, pharmaceutically acceptable salt, solvate, or hydrate thereof. In some embodiments, the pharmaceutical composition comprises from 15 mg/g to 25 mg/g of Compound A, or an isotopic variant, tautomer, prodrug, pharmaceutically acceptable salt, solvate, or hydrate thereof. In some embodiments, the pharmaceutical composition comprises 10 mg/g, 15 mg/g, 20 mg/g, 25 mg/g, or 30 mg/g, of Compound A, or an isotopic variant, tautomer, prodrug, pharmaceutically acceptable salt, solvate, or hydrate thereof. In some embodiments, the pharmaceutical composition comprises 15 mg/g, 16 mg/g, 17 mg/g, 18 mg/g, 19 mg/g, or 20 mg/g, of Compound A, or an isotopic variant, tautomer, prodrug, pharmaceutically acceptable salt, solvate, or hydrate thereof. In some embodiments, the pharmaceutical composition comprises 19.0 mg/g, 19.1 mg/g, 19.2 mg/g, 19.3 mg/g, 19.4 mg/g, 19.5 mg/g, 19.6 mg/g, 19.7 mg/g, 19.8 mg/g, 19.9 mg/g, or 20.0 mg/g of Compound A, or an isotopic variant, tautomer, prodrug, pharmaceutically acceptable salt, solvate, or hydrate thereof.

In some embodiments, the pharmaceutical composition is stable for up to about 7 days at a temperature of from about 20° C. to 25° C. In some embodiments, stability is determined by visual inspection (e.g., precipitation, discoloration) or by HPLC analysis.

In some embodiments, Compound A, or an isotopic variant, tautomer, prodrug, pharmaceutically acceptable salt, solvate, or hydrate thereof, is free of impurities. In some embodiments, Compound A, or an isotopic variant, tautomer, prodrug, pharmaceutically acceptable salt, solvate, or hydrate thereof, is essentially free of impurities. In some embodiments, Compound A, or an isotopic variant, tautomer, prodrug, pharmaceutically acceptable salt, solvate, or hydrate thereof, is substantially free of impurities.

In some embodiments, Compound A, or an isotopic variant, tautomer, prodrug, pharmaceutically acceptable salt, solvate, or hydrate thereof, is at least about 90% pure, at least about 95% pure, at least about 96% pure, at least about 97% pure, at least about 98% pure, or at least about 99% pure. In some embodiments, Compound A, or an isotopic variant, tautomer, prodrug, pharmaceutically acceptable salt, solvate, or hydrate thereof, is at least about 99.0% pure, at least about 99.1% pure, at least about 99.2% pure, at least about 99.3% pure, at least about 99.4% pure, at least about 99.5% pure, at least about 99.6% pure, at least about 99.7% pure, at least about 99.8% pure, or at least about 99.9% pure.

In some embodiments, provided herein is a pharmaceutical composition, comprising:
(i) (S)-1-(3-(5,6-dihydroxypyrimidin-4-yl)-2-(4-((4-(morpholinomethyl)phenyl)ethynyl)phenyl)propyl)azetidine-3-carbonitrile (Compound A):

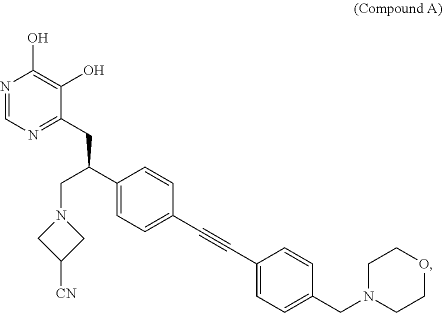 [SMILES: N#CC1CN(C[C@@H](Cc2ncnc(O)c2O)c2ccc(C#Cc3ccc(CN4CCOCC4)cc3)cc2)C1]

or an isotopic variant, tautomer, prodrug, pharmaceutically acceptable salt, solvate, or hydrate thereof; and
(ii) at least one pharmaceutically acceptable excipient;
wherein the pharmaceutical composition is in a dosage form for oral dosing or administration.

In some embodiments, the dosage form is a tablet or a capsule. In some embodiments, the tablet or capsule has an enteric coating. In some embodiments, the dosage form is a tablet. In some embodiments, the dosage form is a capsule.

In other embodiments, the dosage form is a liquid. In some embodiments, the dosage form is a suspension, solution, syrup, or elixir. In some embodiments, the dosage form is a suspension. In some embodiments, the dosage form is a nanosuspension. In some embodiments, the dosage form is a solution.

In some embodiments, Compound A, or an isotopic variant, tautomer, prodrug, pharmaceutically acceptable salt, solvate, or hydrate thereof, is crystalline, microcrystalline, amorphous, or lyophilized.

In some embodiments, Compound A, or an isotopic variant, tautomer, prodrug, pharmaceutically acceptable salt, solvate, or hydrate thereof, is crystalline.

In some embodiments, Compound A, or an isotopic variant, tautomer, prodrug, pharmaceutically acceptable salt, solvate, or hydrate thereof, is amorphous.

In some embodiments, Compound A, or an isotopic variant, tautomer, prodrug, pharmaceutically acceptable salt, solvate, or hydrate thereof, is in an amorphous solid dispersion. In some embodiments, the amorphous solid dispersion is a spray dried dispersion. In some embodiments, the amorphous solid dispersion further comprising a cellulose polymer excipient. In some embodiments, the cellulose polymer excipient comprises cellulose acetate phthalate, carboxymethylcellulosesodium, hydroxypropylcelluloseacetate succinate, hydroxypropyl methylcellulose 606 (HPMC 606), or hydroxypropyl methylcellulose phthalate (HP-55). In some embodiments, the cellulose polymer excipient comprises HPMC 606 or HP-55.

In some embodiments, the pharmaceutical composition is a suspension. In some embodiments, the pharmaceutical composition is a suspension of an amorphous spray dried dispersion of Compound A, or an isotopic variant, tautomer, prodrug, pharmaceutically acceptable salt, solvate, or hydrate thereof.

In some embodiments, the pharmaceutical composition is a nanosuspension. In some embodiments, Compound A, or an isotopic variant, tautomer, prodrug, pharmaceutically acceptable salt, solvate, or hydrate thereof, is nanomilled. In some embodiments, the particles have an average particle size of from about 100 nm to about 750 nm. In some embodiments, the particles have an average particle size of from about 100 nm to about 550 nm. In some embodiments, the particles have an average particle size of from about 150 nm to about 250 nm. In some embodiments, the particles have an average particle size of from about 200 nm to about 250 nm. In some embodiments, the particles have an average particle size of about 200 nm, about 210 nm, about 220 nm, about 230 nm, about 240 nm, or about 250 nm.

In some embodiments, the at least one pharmaceutically acceptable excipient is a co-solvent, oil, surfactant, complexing agent, a solubilizing polymer, a P-gp modulator, a buffering agent, or a combination thereof.

In some embodiments, the co-solvent comprises PEG200, PEG300, PEG400, PEG600, propylene glycol, ethanol, transcutol, glycerin, or a combination thereof. In some embodiments, the cosolvent comprises PEG 400, propylene glycol, transcutol, or a combination thereof.

In some embodiments, the oil comprises sesame oil, soybean oil, vegetable oil, poppyseed oil, safflower oil, peppermint oil, castor oil, oleic acid, maisine CC, capmul MCM, or a combination thereof. In some embodiments, the oil comprises soybean oil, oleic acid, maisine CC, peppermint oil, capmul MCM, or a combination thereof.

In some embodiments, the surfactant comprises polysorbate 20, polysorbate 40, polysorbate 60, polysorbate 80, Gelucire 44/14, vitamin E TPGS, Cremophor RH40, Cremophore RH60, Labrafil M 1944, Labrafil M 2125, Solutol HS 15, or a combination thereof. In some embodiments, the surfactant comprises Gelucire 44/14, vitamin E TPGS, polysorbate 20, polysorbate 80, Labrafil M 1944, Solutol HS 15, or a combination thereof.

In some embodiments, the complexing agent comprises α-cyclodextrin, β-cyclodextrin, γ-cyclodextrin, methyl-β-cyclodextrin (MβCD), (2-hydroxypropyl)-β-cyclodextrin (HPPCD), sulfobutylether-β-cyclodextrin (SBEβCD), or a combination thereof. In some embodiments, the complexing agent comprises HPβCD, SBEβCD, or a combination thereof. In some embodiments, the complexing agent comprises SBEβCD.

In some embodiments, the pharmaceutical composition comprises sulfobutylether-p-cyclodextrin (SBEβCD). In some embodiments, the pharmaceutical composition comprises from about 0.1% to about 50% SBEβCD. In some embodiments, the pharmaceutical composition comprises from about 25% to about 50% SBEβCD. In some embodiments, the pharmaceutical composition comprises about 25%, about 30%, about 35%, about 40%, about 45%, or about 50% SBEβCD. In some embodiments, the pharmaceutical composition comprises about 25% SBEβCD. In some embodiments, the pharmaceutical composition comprises about 30% SBEβCD. In some embodiments, the pharmaceutical composition comprises about 35% SBEβCD. In some embodiments, the pharmaceutical composition comprises about 40% SBEβCD. In some embodiments, the pharmaceutical composition comprises about 45% SBEβCD. In some embodiments, the pharmaceutical composition comprises about 50% SBEβCD. In some embodiments, the pharmaceutical composition comprises from 25% to 50% SBEβCD. In some embodiments, the pharmaceutical composition comprises 25%, 30%, 35%, 40%, 45%, or 50% SBEβCD. In some embodiments, the pharmaceutical composition comprises 25% SBEβCD. In some embodiments, the pharmaceutical composition comprises 30% SBEβCD. In some embodiments, the pharmaceutical composition comprises 35% SBEβCD. In some embodiments, the pharmaceutical composition comprises 40% SBEβCD. In some embodiments, the pharmaceutical composition comprises 45% SBEβCD. In some embodiments, the pharmaceutical composition comprises 50% SBEβCD.

In some embodiments, the solubilizing polymer comprises cellulose acetate phthalate, carboxymethylcellulosesodium, hydroxypropylcelluloseacetate succinate, hydroxypropyl methylcellulose 606 (HPMC 606), hydroxypropyl methylcellulose phthalate (HP-55), polyvinyl caprolactam-polyvinyl acetate-polyethylene glycol graft copolymer (Soluplus, PCL-PVAc-PEG), or poly(propylene oxide)-poly(ethylene oxide) copolymer (Paloxomer). In some embodiments, the solubilizing polymer comprises HPMC 606.

In some embodiments, the pharmaceutical composition comprises hydroxypropyl methylcellulose 606(HPMC 606). In some embodiments, the pharmaceutical composition comprises from about 0.01% to about 10% HPMC 606. In some embodiments, the pharmaceutical composition comprises from about 0.01% to about 5% HPMC 606. In some embodiments, the pharmaceutical composition comprises from about 0.01% to about 1% HPMC 606. In some embodiments, the pharmaceutical composition comprises from about 0.05% to about 0.5% HPMC 606. In some embodiments, the pharmaceutical composition comprises from about 0.05% to about 0.25% HPMC 606. In some embodiments, the pharmaceutical composition comprises about 0.05%, about 0.075%, about 0.1%, about 0.15%, about 0.2%, about 0.25%, about 0.3%, about 0.4%, or about 0.5% HPMC 606. In some embodiments, the pharmaceutical composition comprises from 0.01% to 5% HPMC 606. In some embodiments, the pharmaceutical composition comprises from 0.01% to 1% HPMC 606. In some embodiments, the pharmaceutical composition comprises from 0.05% to 0.5% HPMC 606. In some embodiments, the pharmaceutical composition comprises from 0.05% to 0.25% HPMC 606. In some embodiments, the pharmaceutical composition comprises 0.05%, 0.075%, 0.1%, 0.15%, 0.2%, 0.25%, 0.3%, 0.4%, or 0.5% HPMC 606. In some embodiments, the pharmaceutical composition comprises 0.1% HPMC 606.

In some embodiments, the P-gp modulator comprises vitamin E TPGS or cyclosporin A. In some embodiments, the P-gp modulator comprises vitamin E TPGS.

In some embodiments, the pharmaceutical composition comprises from about 0.1% to about 20% vitamin E TPGS. In some embodiments, the pharmaceutical composition comprises from about 1% to about 10% vitamin E TPGS. In some embodiments, the pharmaceutical composition comprises from about 1% to about 5% vitamin E TPGS. In some embodiments, the pharmaceutical composition comprises about 1.0%, about 1.5%, about 2.0%, about 2.5%, about 3%, about 4%, or about 5% vitamin E TPGS. In some embodiments, the pharmaceutical composition comprises about 2.5% vitamin E TPGS. In some embodiments, the pharmaceutical composition comprises about 5% vitamin E TPGS. In some embodiments, the pharmaceutical composition comprises from 1% to 10% vitamin E TPGS. In some embodiments, the pharmaceutical composition comprises from 1% to 5% vitamin E TPGS. In some embodiments, the pharmaceutical composition comprises 1.0%, 1.5%, 2.0%, 2.5%, 3%, 4%, or 5% vitamin E TPGS. In some embodiments, the pharmaceutical composition comprises 2.5% vitamin E TPGS. In some embodiments, the pharmaceutical composition comprises 5% vitamin E TPGS.

In some embodiments, the buffering agent comprises phosphate, citrate, lactic acid, proline, histidine, or hydroxide, or a combination thereof. In some embodiments, the buffering agent comprises citrate, lactic acid, or a combination thereof. In some embodiments, the buffering agent comprises citrate.

In some embodiments, the pharmaceutical composition has a pH of from about 2.5 to about 11.0. In some embodiments, the pharmaceutical composition has a pH of from about 2.5 to about 7.0. In some embodiments, the pharmaceutical composition has a pH of from about 3.0 to about 4.5. In some embodiments, the pharmaceutical composition has a pH of about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, or about 4.5. In some embodiments, the pharmaceutical composition has a pH of from 2.5 to 11.0. In some embodiments, the pharmaceutical composition has a pH of from 2.5 to 7.0. In some embodiments, the pharmaceutical composition has a pH of from 3.0 to 4.5. In some embodiments, the pharmaceutical composition has a pH of 3.0, 3.1, 3.2, 3.3, 3.4, 3.5, 3.6, 3.7, 3.8, 3.9, 4.0, 4.1, 4.2, 4.3, 4.4, or 4.5.

In some embodiments, the pH of the pharmaceutical composition is adjusted with hydrochloric acid, sodium hydroxide, or a combination thereof. In some embodiments, the pH of the pharmaceutical composition is adjusted with hydrochloric acid. In some embodiments, the pH of the pharmaceutical composition is adjusted with sodium hydroxide.

In some embodiments, the pharmaceutical composition comprises from about 0.1 mg/mL to about 100 mg/mL of Compound A, or an isotopic variant, tautomer, prodrug, pharmaceutically acceptable salt, solvate, or hydrate thereof. In some embodiments, the pharmaceutical composition comprises from about 0.5 mg/mL to about 20 mg/mL of Compound A, or an isotopic variant, tautomer, prodrug, pharmaceutically acceptable salt, solvate, or hydrate thereof. In some embodiments, the pharmaceutical composition comprises from about 1 mg/mL to about 10 mg/mL of Compound A, or an isotopic variant, tautomer, prodrug, pharmaceutically acceptable salt, solvate, or hydrate thereof. In some embodiments, the pharmaceutical composition comprises about 1 mg/mL, about 1.5 mg/mL, about 2 mg/mL, about 2.5 mg/mL, about 3 mg/mL, about 3.5 mg/mL, about 4 mg/mL, about 4.5 mg/mL, or about 5 mg/mL of Compound A, or an isotopic variant, tautomer, prodrug, pharmaceutically acceptable salt, solvate, or hydrate thereof. In some embodiments, the pharmaceutical composition comprises from 0.5 mg/mL to 20 mg/mL of Compound A, or an isotopic variant, tautomer, prodrug, pharmaceutically acceptable salt, solvate, or hydrate thereof. In some embodiments, the pharmaceutical composition comprises from 1 mg/mL to 10 mg/mL of Compound A, or an isotopic variant, tautomer, prodrug, pharmaceutically acceptable salt, solvate, or hydrate thereof. In some embodiments, the pharmaceutical composition comprises 1 mg/mL, 1.5 mg/mL, 2 mg/mL, 2.5 mg/mL, 3 mg/mL, 3.5 mg/mL, 4 mg/mL, 4.5 mg/mL, or 5 mg/mL of Compound A, or an isotopic variant, tautomer, prodrug, pharmaceutically acceptable salt, solvate, or hydrate thereof. In some embodiments, the pharmaceutical composition comprises 3 mg/mL of Compound A, or an isotopic variant, tautomer, prodrug, pharmaceutically acceptable salt, solvate, or hydrate thereof.

In some embodiments, Compound A, or an isotopic variant, tautomer, prodrug, pharmaceutically acceptable salt, solvate, or hydrate thereof, is free of impurities. In some embodiments, Compound A, or an isotopic variant, tautomer, prodrug, pharmaceutically acceptable salt, solvate, or hydrate thereof, is essentially free of impurities. In some embodiments, Compound A, or an isotopic variant, tautomer, prodrug, pharmaceutically acceptable salt, solvate, or hydrate thereof, is substantially free of impurities.

In some embodiments, Compound A, or an isotopic variant, tautomer, prodrug, pharmaceutically acceptable salt, solvate, or hydrate thereof, is at least about 90% pure, at least about 95% pure, at least about 96% pure, at least about 97% pure, at least about 98% pure, or at least about 99% pure. In some embodiments, Compound A, or an isotopic variant, tautomer, prodrug, pharmaceutically acceptable salt, solvate, or hydrate thereof, is at least about 99.0% pure, at least about 99.1% pure, at least about 99.2% pure, at least about 99.3% pure, at least about 99.4% pure, at least about 99.5% pure, at least about 99.6% pure, at least about 99.7% pure, at least about 99.8% pure, or at least about 99.9% pure.

In certain embodiments, Compound A, or an isotopic variant, tautomer, prodrug, pharmaceutically acceptable salt, solvate, or hydrate thereof, is substantially pure, in that it contains less than about 5%, or less than about 1%, or less than about 0.1%, of other organic small molecules, such as unreacted intermediates or synthesis by-products that are created, for example, in one or more of the steps of a synthesis method.

### LpxC, Lipid A and Gram-Negative Bacteria

Metalloproteins influence a vast diversity of biological systems, biological processes, and diseases. For example, UDP-{3-O—[(R)-3-hydroxymyristoyl]}-N-acetylglucosamine deacetylase (LpxC) is an essential enzyme involved in the first committed step in lipid A biosynthesis for gram-negative bacteria. Lipid A is an essential component of the outer membrane of gram-negative bacteria. LpxC is a zinc(II)-dependent metalloenzyme, with two histidines and an aspartic acid residue bound to the zinc(II) ion. Structures of LpxC show the zinc(II) ion is bound to two water molecules, both of which have been implicated in the mechanism of the enzyme. LpxC is highly conserved across strains of gram-negative bacteria, making LpxC an attractive target to treat gram-negative infections.

In recent years, there has been an increase in resistant and multi-drug resistant strains of bacteria. Thus, there is a need for new antibiotics, especially with new mechanisms of action. There remains a need for metalloprotein modulators of LpxC useful in the field of therapeutics, diagnostics, and research.

One embodiment provides a method of inhibiting UDP-{3-O—[(R)-3-hydroxymyristoyl]}-N-acetylglucosamine deacetylase enzyme comprising contacting the enzyme with the LpxC inhibitory compound disclosed herein.

### Methods of Treatment

Disclosed herein are methods of treating disease wherein the inhibition of bacterial growth is indicated. Such disease includes gram-negative bacterial infection. In some embodiments, the method of treating a gram-negative bacterial infection in a patient in need thereof comprises administering to the patient a pharmaceutical composition comprising the LpxC inhibitory compound disclosed herein, or an isotopic variant, tautomer, prodrug, pharmaceutically acceptable salt, solvate, or hydrate thereof, and a pharmaceutically acceptable excipient. In some embodiments, the gram-negative bacterial infection is selected from pneumonia, sepsis, cystic fibrosis, intra-abdominal infection, skin infections and urinary tract infection. In some embodiments, the gram-negative bacterial infection is a urinary tract infection (UTI), a hospital acquired/ventilator-associated pneumonia (HAP/VAP), or an intra-abdominal infection (IAI). In some embodiments, the gram-negative bacterial infection is selected from chronic urinary tract infections, complicated urinary tract infections, cystitis, pyelonephritis, urethritis, recurrent urinary tract infections, bladder infections, urethral infections, and kidney infections. In some embodiments, the compounds described herein are used for the treatment of chronic urinary tract infections. In some embodiments, the compounds described herein are used for the treatment of complicated urinary tract infections. In other embodiments, the compounds described herein are used for the treatment of complicated intra-abdominal infection. In some embodiments, the compounds described herein are used for the treatment of chronic intra-abdominal infection. In other embodiments, the compounds described herein are used for the treatment of hospital acquired pneumonia (HAP) or ventilator associated pneumonia (VAP). In some embodiments the administration is to treat an existing infection. In some embodiments the administration is provided as prophylaxis.

In some embodiments, the LpxC inhibitory compound described herein, or an isotopic variant, tautomer, prodrug, pharmaceutically acceptable salt, solvate, or hydrate thereof, is used for treating conditions caused by the bacterial production of endotoxin and, in particular, by gram-negative bacteria and bacteria that use LpxC in the biosynthesis of lipopolysaccharide (LPS) or endotoxin. In some embodiments, the method of treating a condition caused by endotoxin or LPS in a patient in need thereof comprises administering to the patient a pharmaceutical composition comprising the LpxC inhibitory compound disclosed herein, or an isotopic variant, tautomer, prodrug, pharmaceutically acceptable salt, solvate, or hydrate thereof, and a pharmaceutically acceptable excipient. In another embodiment, the heterocyclic LpxC inhibitory compound and formulations as described herein are useful in the treatment of conditions that are caused or exacerbated by the bacterial production of lipid A and LPS or endotoxin, such as sepsis, septic shock, systemic inflammation, localized inflammation, chronic obstructive pulmonary disease (COPD) and acute exacerbations of chronic bronchitis (AECB). In some embodiments, the method of treating a condition caused by endotoxin or LPS in a patient in need thereof comprises administering to the patient a pharmaceutical composition comprising the LpxC inhibitory compound disclosed herein, or an isotopic variant, tautomer, prodrug, pharmaceutically acceptable salt, solvate, or hydrate thereof, and a pharmaceutically acceptable excipient, wherein the condition caused by endotoxin or LPS is selected from sepsis, septic shock, systemic inflammation, localized inflammation, chronic obstructive pulmonary disease (COPD) and acute exacerbations of chronic bronchitis (AECB).

In other embodiments, the LpxC inhibitory compound described herein, or an isotopic variant, tautomer, prodrug, pharmaceutically acceptable salt, solvate, or hydrate thereof, can be used for the treatment of a serious or chronic respiratory tract infection or complicated urinary tract infections including serious lung and nosocomial infections such as those caused by Enterobacler aerogenes, Enterobacter cloacae, Ewcherichia coli, Klebsiella pnewmnoniae, Klebsiella oxyloca, Kluyvera ascorbata, Kluyvera crocrescense, Shigella sonnei, Proteus mirabilis, Serratia marcescens, Stenotrophmonas maltophilia, Pseudomonas aeruginosa, Burkholderia cepacia, Acinetobacter baumannii. Alcaligenes xylosoxidans, Flavobacterium meningosepticum, Providencia sluarlii and Citrobacter freundii, Haemophilus influenza, Kluyvera species, Legionella species, Moraxella catarrhalis, Enterobacter species, Acinetobacter species, Klebsiella species, Burkholderia species and Proteus species, and infections caused by other bacterial species such as Neisseria species, Shigella species, Salmonella species, Helicobacter pylori, Vibrionaceae and Bordetella species as well as the infections caused by a Brucella species, Francisella tularensis and/or Yersinia pestis.

In one embodiment provided herein is a method of treating a gram-negative bacterial infection in a patient in need thereof comprising administering to the patient a pharmaceutical composition comprising the LpxC inhibitory compound disclosed herein, or an isotopic variant, tautomer, prodrug, pharmaceutically acceptable salt, solvate, or hydrate thereof, and at least one pharmaceutically acceptable excipient.

One embodiment provides a method wherein the gram-negative bacterial infection is selected from pneumonia, sepsis, cystic fibrosis, intra-abdominal infection, skin infection and urinary tract infection.

One embodiment provides a method wherein the gram-negative bacterial infection is selected from chronic urinary tract infection, complicated urinary tract infection, cystitis, pyelonephritis, urethritis, recurrent urinary tract infections, bladder infections, urethral infections and kidney infections.

One embodiment provides a method wherein the gram-negative bacterial infection is chronic urinary tract infections. One embodiment provides a method wherein the gram-negative bacterial infection is complicated urinary tract infections. One embodiment provides a method wherein the administration is to treat an existing infection. One embodiment provides a method wherein the administration is provided as prophylaxis.

In some embodiments, the LpxC inhibitory compound described herein, or an isotopic variant, tautomer, prodrug, pharmaceutically acceptable salt, solvate, or hydrate thereof, is not active against gram-positive bacteria. In some embodiments, the LpxC inhibitory compound described herein, or an isotopic variant, tautomer, prodrug, pharmaceutically acceptable salt, solvate, or hydrate thereof, is not active against Staphylococcus aureus, Enterococcus faecalis, Streptococcus pyogenes, Bacillus thuringiensis, Lactobacillus rhamnosus, Staphylococcus epidermidis, Bifidobacterium brew, Clostridium difficile, Clostridium sordellii, Peptostreptococcus anaerobius, Streptococcus pneumoniae, Corynebacterium jeikeium, Propionibacterium acnes, Listeria monocytogenes, and/or Nocardia cyriacigeorgica complex. Most gut bacteria are Gram-positive, including C. difficile. Therefore, in some embodiments, the lack of activity against gram-positive bacteria is a benefit. In some embodiments, use of the LpxC inhibitory compound described herein, or an isotopic variant, tautomer, prodrug, pharmaceutically acceptable salt, solvate, or hydrate thereof, to treat a gram-negative bacterial infection, as described herein, has no effect on the gut microflora and thus reduces the risk of secondary infections from, for example, C. difficile.

### Combination Therapy

In some instances, Gram-negative bacteria are more resistant to a larger number of antibacterials and chemotherapeutic agents than are gram-positive bacteria due in part to their outer membrane, which acts as an efficient permeability barrier.

A survey of recently reported antibacterials of natural origin showed that over 90% lacked activity against Escherichia coli, although they were active against gram-positive bacteria. Young and Silver (J. Bacteriol. 173(12):3609-14 (1991)) demonstrated that an envA 1 strain, having an altered outer membrane, is sensitive to a variety of large and hydrophobic antibacterials to which wild type E. coli is resistant. Additionally, Vaara, et al., (Antimicrobial Agents and Chemotherapy 37(11):2255-2260 (1993)) review a variety of outer membrane-defective mutants of E. coli and S. typhimurium that show greater susceptibility than the corresponding wild type strain to a variety of antibacterial agents.

In some embodiments, the present invention provides synergistic combinations of antibacterial agents with the LpxC inhibitory compound or pharmaceutical compositions disclosed herein. In some embodiments, the LpxC inhibitory compound disclosed herein has both intrinsic antibacterial properties as well the ability to improve permeability of the outer membrane of gram-negative bacteria to other antibacterial agents. In some embodiments, the antibacterial agent is selected from the group consisting of vancomycin, linezolid, azithromycin, imipenem, teicoplanin, daptomycin, clindamycin, rifampin, cefotaxime, gentamicin, novobiocin, and telavancin.

The use of such synergistic combinations of drugs could have many advantages over conventional single compound therapy, including lowered side-effects of the antibacterial agent due to lower doses used or to shorter time of treatment, more rapid cure of infection shortening hospital stays, increasing spectrum of pathogens controlled, and decreasing incidence of development of resistance to antibiotics.

### Methods of Dosing and Treatment Regimens

In one embodiment, the LpxC inhibitory compound disclosed herein, or an isotopic variant, tautomer, prodrug, pharmaceutically acceptable salt, solvate, or hydrate thereof, are used in the preparation of medicaments for the treatment of diseases or conditions in a mammal that would benefit from modulation of LpxC activity. Methods for treating any of the diseases or conditions described herein in a mammal in need of such treatment, involves administration of pharmaceutical compositions that include the LpxC inhibitory compound disclosed herein, or an isotopic variant, tautomer, prodrug, pharmaceutically acceptable salt, solvate, or hydrate thereof, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

The amount of a given agent that corresponds to such an amount varies depending upon factors such as the particular compound, disease condition and its severity, the identity (e.g., weight, sex) of the subject or host in need of treatment, but nevertheless is determined according to the particular circumstances surrounding the case, including, e.g., the specific agent being administered, the route of administration, the condition being treated, and the subject or host being treated.

In general, however, doses employed for adult human treatment are typically in the range of 0.01 mg-2000 mg per day. In one embodiment, the desired dose is conveniently presented in a single dose or in divided doses administered simultaneously or at appropriate intervals, for example as two, three, four or more sub-doses per day.

In one embodiment, the daily dosages appropriate for the LpxC inhibitory compound disclosed herein, or an isotopic variant, tautomer, prodrug, pharmaceutically acceptable salt, solvate, or hydrate thereof, described herein are from about 0.01 to about 50 mg/kg per body weight. In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the activity of the compound used, the disease or condition to be treated, the mode of administration, the requirements of the individual subject, the severity of the disease or condition being treated, and the judgment of the practitioner.

In any of the aforementioned aspects are further embodiments in which the effective amount of the LpxC inhibitory compound disclosed herein, or an isotopic variant, tautomer, prodrug, pharmaceutically acceptable salt, solvate, or hydrate thereof, is: (a) systemically administered to the mammal; and/or (b) administered orally to the mammal.

In some embodiments, the LpxC inhibitory compound disclosed herein, or an isotopic variant, tautomer, prodrug, pharmaceutically acceptable salt, solvate, or hydrate thereof, is administered is dose selected from about 25 mg, about 50 mg, about 75 mg, about 100 mg, about 125 mg, about 150 mg, about 175 mg, about 200 mg, about 225 mg, about 250 mg, about 275 mg, about 300 mg, about 325 mg, about 350 mg, about 375 mg, and about 400 mg. In some embodiments, the dose is administered once a day. In some embodiments, the dose is administered twice a day.

### Articles of Manufacture and Kits

Disclosed herein, in certain embodiments, are kits and articles of manufacture for use with one or more methods described herein. In some embodiments, additional components of the kit comprises a carrier, package, or container that is compartmentalized to receive one or more containers such as vials, tubes, and the like, each of the container(s) comprising one of the separate elements to be used in a method described herein. Suitable containers include, for example, bottles, vials, plates, syringes, and test tubes. In one embodiment, the containers are formed from a variety of materials such as glass or plastic.

The articles of manufacture provided herein contain packaging materials. Examples of pharmaceutical packaging materials include, but are not limited to, bottles, tubes, bags, containers, and any packaging material suitable for a selected formulation and intended mode of use.

For example, the container(s) include one or more of the compounds described herein. Such kits optionally include an identifying description or label or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

Other embodiments and uses will be apparent to one skilled in the art in light of the present disclosures. The following examples are provided merely as illustrative of various embodiments and shall not be construed to limit the invention in any way.

### EXAMPLES

### I. Biological Evaluation

### Example 1: Bacterial Susceptibility Testing

Minimal inhibitory concentrations (MIC) against a variety of gram-negative and gram-positive bacterial strains were determined by the broth microdilution method in accordance with the Clinical and Laboratory Standards Institute (CLSI) guidelines. In brief, organism suspensions were adjusted to a 0.5 McFarland standard to yield a final inoculum between 3×10⁵ and 7×10⁵ colony-forming units (CFU)/mL. Drug dilutions and inocula were made in sterile, cation adjusted Mueller-Hinton Broth (Beckton Dickinson). An inoculum volume of 100 μL was added to wells containing 100 μL. of broth with 2-fold serial dilutions of drug. All inoculated microdilution trays were incubated in ambient air at 35° C. for 18-24 h. Following incubation, the lowest concentration of the drug that prevented visible growth (OD600 nm<0.05) was recorded as the MIC. Performance of the assay was monitored by the use of laboratory quality-control strains and levofloxacin, a compound with a defined MIC spectrum, in accordance with CLSI guidelines.

Exemplary in vitro assay data against select bacteria for Compound A, Meropenem, and Levofloxacin is provided in Table 1.

TABLE 1

| | | | MIC (μg/mL) | |
|---|---|---|---|---|
| Bacterium | Strain | Compound A | Meropenem | Levofloxacin |
| E. coli | ATCC 25922 | 0.5 | 0.03 | 0.03 |
| K. pneumoniae | ATCC 13883 | 0.5 | | |
| S. aureus | ATCC 29213 | >64 | 0.125 | 0.25 |
| E. faecalis | ATCC 29212 | >64 | >1 | 1 |
| S. pyogenes | ATCC 12384 | >64 | 0.008 | 0.5 |
| B. thuringiensis | ATCC 35646 | >64 | 0.06 | 0.125 |
| L. rhamnosus | ATCC 53103 | >64 | >1 | 1 |
| S. epidermidis | ATCC 35984 | >64 | >1 | 0.125 |
| B. breve | HM 412 | >64 | >1 | 4 |
| C. difficile | ATCC 700057 | >64 | 1 | 4 |
| C. sordellii | ATCC 9714 | >64 | 0.015 | 1 |
| P. anaerobius | DSM 20357 | >64 | >1 | 0.5 |
| S. pneumoniae | ATCC 49619 | >64 | 0.06 | 1 |
| C. jeikeium | NCTC 11914 | >64 | >1 | 1 |
| P. acnes | ATCC 6919 | >64 | 0.06 | 0.5 |
| L. monocytogenes | ATCC 7644 | >64 | 0.125 | 1 |
| N. cyriacigeorgica complex | NEQAS 3295 | >64 | >1 | 8 |

Compound A has high selectivity for gram-negative bacteria over gram-positive bacteria. Standard of care antibiotics Meropenem and Levofloxacin, in contrast, do have activity against various strains of gram-positive bacteria.

### II. Pharmaceutical Compositions

### Example 2: Intravenous (I.V.) Solution Formulation

Compound A is formulated as a solution at a target concentration of 20 mg/g of Compound A. The formulation comprises Compound A, SBEβCD (Captisol, sulfobutylether-β-cyclodextrin) or HPβCD (2-hydroxypropyl-β-cyclodextrin), hydrochloric acid (as needed), sodium hydroxide (as needed), and water. Compound A is added to an aqueous solution of SBEβCD or HPβCD, and the pH adjusted to 4.2±0.1 using hydrochloric acid/or sodium hydroxide. The Compound A solution is then filtered through a 0.2 μm membrane filter to yield the final solution formulation.

Table 2 describes the composition of Compound A intravenous solution formulations, at about 20 mg/g.

TABLE 2

| | | Compound A | | |
|---|---|---|---|---|
| | | concentration | | Osmolarity |
| Formulation | Excipient | [mg/g]^{a} | pH | [mOsm/Kg] |
| 3A | 2.5% SBEβCD | 16.98 | 4.21 | 584 |
| 3B | 5% SBEβCD | 18.16 | 4.25 | 796 |
| 3C | 10% SBEβCD | 19.34 | 4.26 | 854 |
| 3D | 2.5% HPβCD | 19.03 | 4.29 | 527 |
| 3E | 5% HPβCD | 17.62 | 4.27 | 599 |
| 3F | 10% HPβCD | 19.06 | 4.24 | 659 |

^{a}as determined by HPLC.

Turbid solutions are observed for all formulations.

The solution formulations can be stored at ambient temperature for up to 1 week with no visible changes.

### Example 3: Precipitation Studies for I.V. Formulations

In order to model the behavior of the formulations on injection into the bloodstream, the I.V. solution formulations from Example 4 were diluted into PBS buffer (pH 7.4) [9 parts PBS: 1 part formulation]. After 15 minutes, the samples are analyzed by HPLC to determine the amount of compound in solution and visual observation. Table 3 shows the results of the precipitation studies.

TABLE 3

| | | Compound A | |
|---|---|---|---|
| | | concentration | |
| Formulation | Excipient | [mg/g]^{a} | pH |
| 3A | 2.5% SBEβCD | 1.26 | 6.41 |
| 3B | 5% SBEβCD | 2.35 | 6.39 |
| 3C | 10% SBEβCD | 7.12 | 6.48 |
| 3D | 2.5% HPβCD | 1.01 | 6.35 |
| 3E | 5% HPβCD | 1.59 | 6.33 |
| 3F | 10% HPβCD | 5.29 | 6.45 |

^{a}as determined by HPLC.

Visually, higher concentrations of excipient led to less precipitation. This is confirmed by the HPLC analysis. The final pH for each formulation was approximately 6.4.

### Example 4: Oral Solution Formulation Excipient Screening

The solubility of Compound A was estimated by HPLC in multiple potential excipients for a solution formulation (Table 4). Low solubility was observed for most excipients, although acidifying the excipients led to higher solubility.

TABLE 4

| | | Estimated |
|---|---|---|
| | | solubility |
| Excipient | Category | [mg/mL] |
| PEG400 | Co-solvent | <10 |
| Propylene glycol | | <10 |
| Propylene glycol (pH 0.5) | | ≥45 |
| Transcutol | | <10 |
| Transcutol (pH 0.5) | | ≥10 |
| Gelucire 44/14 | Surfactants | <10 |
| Vitamin E TPGS | | <10 |
| Cremophor RH40 | | 0.90 |
| Polysorbate 20 | | 0.85 |
| Polysorbate 80 | | 0.62 |
| Labrafil M 1944 | | 0.12 |
| Solutol HS 15 | | 0.63 |
| Soybean oil | Oils | 0.02 |
| Oleic acid | | 0.48 |
| Maisine CC | | 0.13 |
| Peppermint oil | | 0.05 |
| Capmul MCM | | 0.33 |
| SBEβCD (30% w/v), pH 3 | Complexing agents | <10 |
| SBEβCD (30% w/v), pH 0.5 | | ≥45 |
| Lactic acid | Acids | ≥20 |
| HCl solution (pH 0.5) | | ≥45 |

### Example 5: Oral Solution Formulations

Compound A is formulated as a solution at a target concentration of ca. 40-45 mg/g of Compound A. A stock solutions of ca. 75 mg/g Compound A in a 2:1 mixture of PEG400:propylene glycol, pH 0.5 was diluted into aqueous vehicles to arrive at the final solution formulations. After addition of the stock solution, the pH of the formulation was adjusted to ca. 3.0-4.0 using NaOH. No precipitation was observed upon pH adjustment.

Table 5 describes the composition of Compound A solution formulations, at about 40-45 mg/g.

TABLE 5

| Formulation | Formulation Composition | pH |
|---|---|---|
| 6A | 40% PEG400, 20% PG, 5% Vitamin E | 3.36 |
| | TPGS | |
| 6B | 40% PEG400, 20% PG, 5% Vitamin E | 3.21 |
| | TPGS + 0.1% HPMC 606 | |
| 6C | 40% PEG400, 20% PG, 5% Vitamin E | 2.98 |
| | TPGS + 0.1% Soluplus | |
| 6D | 40% PEG400, 20% PG, 10% SBEβCD | 3.67 |
| 6E | 40% PEG400, 20% PG, 10% SBEβCD + | 3.10 |
| | 0.1% HPMC 606 | |
| 6F | 40% PEG400, 20% PG, 10% SBEβCD + | 3.63 |
| | 0.1% Poloxamer 407 | |
| 6G | 40% PEG400, 20% PG, 20% SBEβCD | 3.96 |

Additional formulations of Compound A at a target concentration of ca. 40-45 mg/g of Compound A were prepared as per Table 6 by dissolving Compound A directly into the vehicle.

TABLE 6

| Formulation | Formulation Composition |
|---|---|
| 6H | 30% SBEβCD + 0.1% HPMC 606 |
| 6I | 0.1M HCl |

### Example 6: Precipitation Studies for Oral Formulations

In order to model the behavior of the formulations on administration into the GI system, the oral solution formulations from Example 7 were diluted into FaSSIF buffer (pH 6.1) [1 part FaSSIF: 1 part formulation], and were incubated at 37° C. for 50 min with stirring at 200 rpm. The samples are analyzed by UV-HPLC to determine the amount of compound in solution and visual observation. Table 7 shows the results of the precipitation studies. The formulation containing 30% SBEβCD and 0.1% HPMC 606 showed the least amount of precipitation.

TABLE 7

| | | Compound A |
|---|---|---|
| | | concentration |
| Formulation | Formulation Composition | [mg/g]^{a} |
| 6A | 40% PEG400, 20% PG, 5% Vitamin | 0.89 |
| | E TPGS | |
| 6B | 40% PEG400, 20% PG, 5% Vitamin | 1.01 |
| | E TPGS + 0.1% HPMC 606 | |
| 6C | 40% PEG400, 20% PG, 5% Vitamin | 0.73 |
| | E TPGS + 0.1% Soluplus | |
| 6D | 40% PEG400, 20% PG, 10% SBEβCD | 1.38 |
| 6E | 40% PEG400, 20% PG, 10% SBEβCD + | 3.00 |
| | 0.1% HPMC 606 | |
| 6F | 40% PEG400, 20% PG, 10% SBEβCD + | 1.50 |
| | 0.1% Poloxamer 407 | |
| 6G | 40% PEG400, 20% PG, 20% SBEβCD | 13.43 |
| 6H | 30% SBEβCD + 0.1% HPMC 606 | 31.2 |
| 6I | 0.1M HCl | 0.17 |

^{a}as determined by HPLC.

### Example 7: Amorphous Solid Dispersions

### Preparation of Spray Dried Dispersions (SDDs)

Selected polymer excipients are dissolved in TH-F:water (9:1) at a concentration of 8 mg/mL. After complete solubilization, Compound A is added to the previous solution at a concentration of 4 mg/mL to achieve approximate drug loading of 33%. Spray-drying was performed using 4 MB Trix spray dryer (ProCepT) with the following settings:

| Parameter | Condition | |
|---|---|---|
| Inlet temperature | 100° | C. |
| Outlet temperature | ~62-64° | C. |
| Nozzle gauge | 1.0 | mm |
| Airspeed | 0.35 | m³/min |
| Nozzle air flow rate | ~10 | L/min |
| Flow rate | 3 | mL/min |

Yields for the two polymers are presented in the following table:

| Polymer Excipient | Yield (%) |
|---|---|
| HPMC 606 | 76.3 |
| HP-55 | 84.3 |

X-Ray Powder Diffractograms (XRPDs) for each of the SDD demonstrated a lack of crystallinity with no characteristic peaks observed.

### Stability of Spray Dried Dispersions (SDDs)

The resulting SDDs were stored for two weeks at either 25° C. under ambient conditions (25° C./Amb), or 40° C. at 75% relative humidity (RH) (40° C./75% RH) and were analysed by HPLC and XRPD to determine stability of the dispersion, as shown in Table 8.

TABLE 8

| | HPMC 606 1:2 SDD | | | HP-55 1:2 SDD | | |
|---|---|---|---|---|---|---|
| | (33.6% drug load) | | | (32.6% drug load) | | |
| Assay | % in | % peak | | % in | % peak | |
| Time Point | SDD | purity | XRPD | SDD | purity | XRPD |
| 0 h | 100 | 96.9 | amorphous | 100 | 96.6 | amorphous |
| 2 weeks | 92.0 | 95.4 | amorphous | 96.3 | 95.2 | amorphous |
| (25° C./Amb) | | | | | | |
| 2 weeks | 91.4 | 93.5 | slight | 66.6 | 72.1 | amorphous |
| (40° C./75% RH) | | | crystallinity | | | |

Chemical instability was observed in both SDDs upon storage at 40° C./75% RH conditions. Instability was more prominent for HP-55 SDD.

### Example 8: Oral SDD Suspension Formulation

This example describes the preparation of a suspension formulation comprising a suspension vehicle and a spray-dried dispersion formulation of Compound A (Example 9).

The SDD formulation of Example 9 was generated by spray drying, containing 33% by weight of Compound A, 66% by weight of HPMC 606.

A suspension vehicle was formed by dissolving vitamin E TPGS in a citrate buffer at loading of 2.5% vitamine E TPGS.

The SDD containing Compound A was dispersed throughout the vehicle at a particle concentration of 3 mg/mL.

### Example 9: Oral Nanosuspension Formulation

This example describes the preparation of a nanosuspension formulation comprising a suspension vehicle and a nanomilled particles of Compound A.

A suspension vehicle was formed by dissolving vitamin E TPGS in water at loading of 2.5% vitamine E TPGS.

Compound A was added to the suspension vehicle at a particle concentration of 3 mg/mL. The suspension was nanomilled for 1.5 h at 600 rpm using a Fritsch planetary mill. A mono-dispersed nanosuspension with PDI of 0.18 and size of 210 nm was achieved.

### III. Pharmacokinetics

### Example 10: Oral PK (Rat)

Male Rats were acclimated to dosing (e.g., oral gavage) 2-3 times prior to the study. On the day of the study, food was removed for 5-6 hours, then the mice were dosed with test article (e.g., by oral gavage at a volume of 10 mL/kg). Blood was collected up to 8-times serially and analyzed for presence of Compound A. Results for exemplary oral formulations are shown in Table 9.

TABLE 9

| | No. of | Dose | Cₘₐₓ | CV^{b} | AUClas | CV^{c} |
|---|---|---|---|---|---|---|
| Formulation^{a} | Animals | [mg/kg] | [ng/mL] | [%] | [ng × h/mL]t | [%] |
| A | 3 | 30 | 417 | 33.5 | 1780 | 18.7 |
| B | 3 | 30 | 2540 | 10.8 | 5620 | 10.4 |
| C | 3 | 30 | 1940 | 21.9 | 5330 | 11.1 |

^{a}Formulation Compositions
A: solution in 30% w/v SBEβCD + 0.1% w/v HPMC606
B: SDD Suspension in 2.5% w/v Vitamin E TPGS in citrate buffer
C: nanosuspension in 2.5% w/v Vitamin E TPGS
^{b}inter-animal variability of Cₘₐₓ
^{c}inter-animal variability of AUCₗₐₛₜ

Systemic exposure was higher in the animals dosed with SDD suspension or nanosuspension formulations and those dosed with the solution formulation.
The examples and embodiments described herein are for illustrative purposes only and various modifications or changes suggested to persons skilled in the art are to be included within the spirit and purview of this application and scope of the appended claims.

What is claimed is:

1. A pharmaceutical composition, comprising:
(i) (S)-1-(3-(5,6-dihydroxypyrimidin-4-yl)-2-(4-((4-(morpholinomethyl)phenyl)ethynyl)phenyl)propyl)azetidine-3-carbonitrile (Compound A): 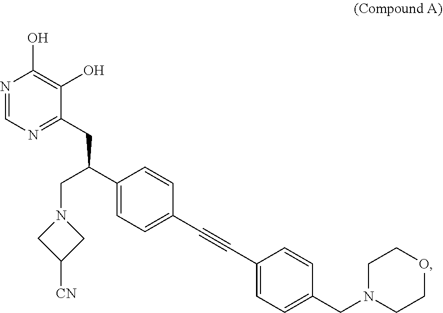 [SMILES: N#CC1CN(C[C@@H](Cc2ncnc(O)c2O)c2ccc(C#Cc3ccc(CN4CCOCC4)cc3)cc2)C1]
or an isotopic variant, tautomer, prodrug, pharmaceutically acceptable salt, solvate, or hydrate thereof; and
(ii) at least one pharmaceutically acceptable excipient.

2. The pharmaceutical composition of claim 1, wherein the pharmaceutical composition is in a dosage form for dosing or administration by injection.

3. The pharmaceutical composition of claim 2, wherein the pharmaceutical composition is in a dosage form for intravenous (I.V.) injection or infusion, or intramuscular, subcutaneous, or intradermal injection.

4. The pharmaceutical composition of claim 2, wherein the pharmaceutical composition is in a dosage form for I.V. injection or infusion.

5. The pharmaceutical composition of any one of claims 2-4, wherein the pharmaceutical composition is a solution.

6. The pharmaceutical composition of any one of claims 1-5, wherein Compound A, or an isotopic variant, tautomer, prodrug, pharmaceutically acceptable salt, solvate, or hydrate thereof, is crystalline, microcrystalline, amorphous, or lyophilized.

7. The pharmaceutical composition of claim 6, wherein Compound A, or an isotopic variant, tautomer, prodrug, pharmaceutically acceptable salt, solvate, or hydrate thereof, is crystalline.

8. The pharmaceutical composition of claim 6, wherein Compound A, or an isotopic variant, tautomer, prodrug, pharmaceutically acceptable salt, solvate, or hydrate thereof, is amorphous.

9. The pharmaceutical composition of any one of claims 1-8, wherein the at least one pharmaceutically acceptable excipient is a co-solvent, oil, surfactant, complexing agent, a solubilizing polymer, a P-gp modulator, a buffering agent, or a combination thereof.

10. The pharmaceutical composition of any one of claims 1-9, wherein the at least one pharmaceutically acceptable excipient is a complexing agent.

11. The pharmaceutical composition of claim 10, wherein the complexing agent comprises α-cyclodextrin, β-cyclodextrin, γ-cyclodextrin, methyl-β-cyclodextrin (MOCD), (2-hydroxypropyl)-β-cyclodextrin (HPβCD), sulfobutylether-β-cyclodextrin (SBEβCD), or a combination thereof.

12. The pharmaceutical composition of any one of claims 1-11, wherein the pharmaceutical composition comprises sulfobutylether-β-cyclodextrin (SBEβCD).

13. The pharmaceutical composition of claim 12, wherein the pharmaceutical composition comprises from about 1% to about 20% sulfobutylether-β-cyclodextrin (SBEβCD).

14. The pharmaceutical composition of claim 12, wherein the pharmaceutical composition comprises from about 2.5% to about 10% sulfobutylether-β-cyclodextrin (SBEβCD).

15. The pharmaceutical composition of claim 12, wherein the pharmaceutical composition comprises about 2.5%, about 5%, or about 10% sulfobutylether-β-cyclodextrin (SBEβCD).

16. The pharmaceutical composition of any one of claims 1-15, wherein the pharmaceutical composition has a pH of from about 2.5 to about 11.0.

17. The pharmaceutical composition of any one of claims 1-16, wherein the pharmaceutical composition has a pH of from about 2.5 to about 7.0.

18. The pharmaceutical composition of any one of claims 1-17, wherein the pharmaceutical composition has a pH of from about 4.0 to about 5.0.

19. The pharmaceutical composition of any one of claims 1-18, wherein the pharmaceutical composition has a pH of about 4.0, about 4.1, about 4.2, about 4.3, about 4.4, about 4.5, about 4.6, about 4.7, about 4.8, about 4.9, or about 5.0.

20. The pharmaceutical composition of anyone of claims 1-19, wherein the pH of the pharmaceutical composition is adjusted with hydrochloric acid, sodium hydroxide, or a combination thereof.

21. The pharmaceutical composition of any one of claims 1-20, wherein the pharmaceutical composition comprises from about 0.1 mg/mL to about 100 mg/mL of Compound A, or an isotopic variant, tautomer, prodrug, pharmaceutically acceptable salt, solvate, or hydrate thereof.

22. The pharmaceutical composition of any one of claims 1-21, wherein the pharmaceutical composition comprises from about 10 mg/mL to about 50 mg/mL of Compound A, or an isotopic variant, tautomer, prodrug, pharmaceutically acceptable salt, solvate, or hydrate thereof.

23. The pharmaceutical composition of any one of claims 1-22, wherein the pharmaceutical composition comprises from about 15 mg/mL to about 35 mg/mL of Compound A, or an isotopic variant, tautomer, prodrug, pharmaceutically acceptable salt, solvate, or hydrate thereof.

24. The pharmaceutical composition of any one of claims 1-22, wherein the pharmaceutical composition comprises about 10 mg/mL, about 15 mg/mL, about 20 mg/mL, about 25 mg/mL, about 30 mg/mL, about 35 mg/mL, about 40 mg/mL, about 45 mg/mL, or about 50 mg/mL of Compound A, or an isotopic variant, tautomer, prodrug, pharmaceutically acceptable salt, solvate, or hydrate thereof.

25. The pharmaceutical composition of any one of claims 1-20, wherein the pharmaceutical composition comprises from about 15 mg/g to about 25 mg/g of Compound A, or an isotopic variant, tautomer, prodrug, pharmaceutically acceptable salt, solvate, or hydrate thereof.

26. The pharmaceutical composition of anyone of claims 1-20 or 25, wherein the pharmaceutical composition comprises about 10 mg/g, about 15 mg/g, about 20 mg/g, about 25 mg/g, or about 30 mg/g of Compound A, or an isotopic variant, tautomer, prodrug, pharmaceutically acceptable salt, solvate, or hydrate thereof.

27. The pharmaceutical composition of any one of claims 1-26, wherein the pharmaceutical composition is stable for up to about 7 days at a temperature of from about 20° C. to 25° C.

28. The pharmaceutical composition of claim 1, wherein the pharmaceutical composition is in a dosage form for oral dosing or administration.

29. The pharmaceutical composition of claim 28, wherein the dosage form is a liquid.

30. The pharmaceutical composition of claim 29, wherein the dosage form is a suspension, solution, syrup, or elixir.

31. The pharmaceutical composition of claim 29 or 30, wherein the dosage form is a suspension.

32. The pharmaceutical composition of any one of claims 29-31, wherein the dosage form is a nanosuspension.

33. The pharmaceutical composition of claim 29 or 30, wherein the dosage form is a solution.

34. The pharmaceutical composition of claim 28, wherein the dosage form is a tablet or a capsule.

35. The pharmaceutical composition of anyone of claims 1 or 28-34, wherein Compound A, or an isotopic variant, tautomer, prodrug, pharmaceutically acceptable salt, solvate, or hydrate thereof, is crystalline, microcrystalline, amorphous, or lyophilized.

36. The pharmaceutical composition of claim 35, wherein Compound A, or an isotopic variant, tautomer, prodrug, pharmaceutically acceptable salt, solvate, or hydrate thereof, is crystalline.

37. The pharmaceutical composition of claim 35, wherein Compound A, or an isotopic variant, tautomer, prodrug, pharmaceutically acceptable salt, solvate, or hydrate thereof, is amorphous.

38. The pharmaceutical composition of any one of claims 1 or 28-34, wherein Compound A, or an isotopic variant, tautomer, prodrug, pharmaceutically acceptable salt, solvate, or hydrate thereof, is in an amorphous solid dispersion.

39. The pharmaceutical composition of claim 38, wherein the amorphous solid dispersion is a spray dried dispersion.

40. The pharmaceutical composition of claim 38 or 39, wherein the amorphous solid dispersion further comprising a cellulose polymer excipient.

41. The pharmaceutical composition of claim 40, wherein the cellulose polymer excipient comprises cellulose acetate phthalate, carboxymethylcellulose sodium, hydroxypropylcellulose acetate succinate, hydroxypropyl methylcellulose 606(HPMC 606), or hydroxypropyl methylcellulose phthalate (HP-55).

42. The pharmaceutical composition of anyone of claims 1 or 28-41, wherein the at least one pharmaceutically acceptable excipient is a co-solvent, oil, surfactant, complexing agent, a solubilizing polymer, a P-gp modulator, a buffering agent, or a combination thereof.

43. The pharmaceutical composition of claim 42, wherein the co-solvent comprises PEG200, PEG300, PEG400, PEG600, propylene glycol, ethanol, transcutol, glycerin, or a combination thereof.

44. The pharmaceutical composition of claim 42, wherein the oil comprises sesame oil, soybean oil, vegetable oil, poppyseed oil, safflower oil, peppermint oil, castor oil, oleic acid, maisine CC, capmul MCM, or a combination thereof.

45. The pharmaceutical composition of claim 42, wherein the surfactant comprises polysorbate 20, polysorbate 40, polysorbate 60, polysorbate 80, Gelucire 44/14, vitamin E TPGS, Cremophor RH40, Cremophore RH60, Labrafil M 1944, Labrafil M 2125, Solutol HS 15, or a combination thereof.

46. The pharmaceutical composition of claim 42, wherein the complexing agent comprises α-cyclodextrin, β-cyclodextrin, 7-cyclodextrin, methyl-β-cyclodextrin (MβCD), (2-hydroxypropyl)-β-cyclodextrin (HPPCD), sulfobutylether-β-cyclodextrin (SBEβCD), or a combination thereof.

47. The pharmaceutical composition of claim 42, wherein the solubilizing polymer comprises cellulose acetate phthalate, carboxymethylcellulose sodium, hydroxypropylcellulose acetate succinate, hydroxypropyl methylcellulose 606 (HPMC 606), hydroxypropyl methylcellulose phthalate (HP-55), polyvinyl caprolactam-polyvinyl acetate-polyethylene glycol graft copolymer (Soluplus, PCL-PVAc-PEG), or poly(propylene oxide)-poly(ethylene oxide) copolymer (Paloxomer).

48. The pharmaceutical composition of claim 42, wherein the P-gp modulator comprises vitamin E TPGS or cyclosporin A.

49. The pharmaceutical composition of claim 42, wherein the buffering agent comprises phosphate, citrate, lactic acid, proline, histidine, or hydroxide, or a combination thereof.

50. The pharmaceutical composition of any one of claims 1 or 28-49, wherein the pharmaceutical composition comprises sulfobutylether-β-cyclodextrin (SBEβCD).

51. The pharmaceutical composition of claim 50, wherein the pharmaceutical composition comprises from about 25% to about 50% sulfobutylether-β-cyclodextrin (SBEβCD).

52. The pharmaceutical composition of claim 50 or 51, wherein the pharmaceutical composition comprises about 25%, about 30%, about 35%, about 40%, about 45%, or about 50% sulfobutylether-β-cyclodextrin (SBEβCD).

53. The pharmaceutical composition of any one of claims 1 or 28-52, wherein the pharmaceutical composition comprises HPMC 606.

54. The pharmaceutical composition of claim 53, wherein the pharmaceutical composition comprises from about 0.05% to about 0.5% HPMC606.

55. The pharmaceutical composition of claim 53 or 54, wherein the pharmaceutical composition comprises about 0.05%, about 0.075%, about 0.1%, about 0.15%, about 0.2%, about 0.25%, about 0.3%, about 0.4%, or about 0.5% HPMC606.

56. The pharmaceutical composition of any one of claims 1 or 28-49, wherein the pharmaceutical composition comprises vitamin E TPGS.

57. The pharmaceutical composition of claim 56, wherein the pharmaceutical composition comprises from about 1% to about 10% vitamin E TPGS.

58. The pharmaceutical composition of claim 56 or 57, wherein the pharmaceutical composition comprises about 1.0%, about 1.5%, about 2.0%, about 2.5%, about 3%, about 4%, or about 5% vitamin E TPGS.

59. The pharmaceutical composition of any one of claims 1 or 28-58, wherein the pharmaceutical composition has a pH of from about 2.5 to about 11.0.

60. The pharmaceutical composition of any one of claims 1 or 28-59, wherein the pharmaceutical composition has a pH of from about 2.5 to about 7.0.

61. The pharmaceutical composition of any one of claims 1 or 28-60, wherein the pharmaceutical composition has a pH of from about 3.0 to about 4.5.

62. The pharmaceutical composition of any one of claims 1 or 28-61, wherein the pharmaceutical composition has a pH of about 3.0, about 3.1, about 3.2, about 3.3, about 3.4, about 3.5, about 3.6, about 3.7, about 3.8, about 3.9, about 4.0, about 4.1, about 4.2, about 4.3, about 4.4, or about 4.5.

63. The pharmaceutical composition of any one of claims 1 or 28-62, wherein the pH of the pharmaceutical composition is adjusted with hydrochloric acid and/or sodium hydroxide.

64. The pharmaceutical composition of any one of claims 1 or 28-63, wherein the pharmaceutical composition comprises from about 0.1 mg/mL to about 100 mg/mL of Compound A, or an isotopic variant, tautomer, prodrug, pharmaceutically acceptable salt, solvate, or hydrate thereof.

65. The pharmaceutical composition of any one of claims 1 or 28-64, wherein the pharmaceutical composition comprises from about 0.5 mg/mL to about 20 mg/mL of Compound A, or an isotopic variant, tautomer, prodrug, pharmaceutically acceptable salt, solvate, or hydrate thereof.

66. The pharmaceutical composition of any one of claims 1 or 28-65, wherein the pharmaceutical composition comprises from about 1 mg/mL to about 10 mg/mL of Compound A, or an isotopic variant, tautomer, prodrug, pharmaceutically acceptable salt, solvate, or hydrate thereof.

67. The pharmaceutical composition of any one of claims 1 or 28-66, wherein the pharmaceutical composition comprises about 1 mg/mL, about 1.5 mg/mL, about 2 mg/mL, about 2.5 mg/m L, about 3 mg/mL, about 3.5 mg/mL, about 4 mg/mL, about 4.5 mg/mL, or about 5 mg/mL of Compound A, or an isotopic variant, tautomer, prodrug, pharmaceutically acceptable salt, solvate, or hydrate thereof.

68. The pharmaceutical composition of any one of claims 1-67, wherein Compound A, or an isotopic variant, tautomer, prodrug, pharmaceutically acceptable salt, solvate, or hydrate thereof, is substantially free of impurities.

69. The pharmaceutical composition of any one of claims 1-68, wherein Compound A, or an isotopic variant, tautomer, prodrug, pharmaceutically acceptable salt, solvate, or hydrate thereof, is at least about 90% pure, at least about 95% pure, at least about 96% pure, at least about 97% pure, at least about 98% pure, or at least about 99% pure.

70. A method of treating a gram-negative bacterial infection in a patient in need thereof comprising administering to the patient the pharmaceutical composition of any one of claims 1-69.

71. The method of claim 70, wherein the gram-negative bacterial infection is selected from pneumonia, sepsis, cystic fibrosis, intra-abdominal infection, skin infection and urinary tract infection.

72. The method of claim 70, wherein the gram-negative bacterial infection is selected from chronic urinary tract infection, complicated urinary tract infection, cystitis, pyelonephritis, urethritis, recurrent urinary tract infections, bladder infections, urethral infections and kidney infections.

73. The method of any one of claims 70-72, wherein the gram-negative bacterial infection is chronic urinary tract infections.

74. The method of any one of claims 70-72, wherein the gram-negative bacterial infection is complicated urinary tract infections.

75. The method of any one of claims 70-74, wherein the composition has no effect on gram-positive bacteria.

76. The method of any one of claims 70-75, wherein the composition is administered to the patient by I.V. injection or infusion.

77. The method of any one of claims 70-75, wherein the composition is administered to the patient orally.

78. The method of any one of claims 70-77, wherein the administration is to treat an existing infection.

79. The method of any one of claims 70-77, wherein the administration is provided as prophylaxis.
